(19) United States
(12) Patent Application Publication
(10) Pub. No.: US 2024/0417477 A1
(43) Pub. Date: Dec. 19, 2024

(54) ANTIBODY CONJUGATES FOR THE TREATMENT OF CANCER

(71) Applicant: B.G. Negev Technologies and Applications Ltd., at Ben-Gurion University, Beer Sheva (IL)

(72) Inventors: Moshe ELKABETS, Beit Kama (IL); Amir AHARONI, Beit Kama (IL); Ksenia YEGODAYEV MEIR, Yeruham (IL)

(21) Appl. No.: 18/757,149

(22) Filed: Jun. 27, 2024

Related U.S. Application Data
(63) Continuation-in-part of application No. PCT/IL2022/051400, filed on Dec. 28, 2022.

(30) Foreign Application Priority Data
Dec. 30, 2021 (IL) 289525

(51) Int. Cl. C07K 16/28; A61K 39/00; A61P 35/00

(52) U.S. Cl. CPC C07K 16/2863; A61P 35/00; C07K 16/2818; A61K 2039/505; C07K 2317/31; C07K 2317/52; C07K 2317/565; C07K 2317/622; C07K 2317/76; C07K 2317/92

(57) ABSTRACT

Inhibitory bi-specific antibody-like constructs comprising two different single-chain variable fragments (scFv), one targeted against the receptor AXL and one against programmed cell death protein 1 (PD-1) are provided. These bi-specific constructs simultaneously target AXL and PD-1 on cancer and/or immune cells and effectively inhibit tumor growth. Compositions comprising the bi-specific molecules and their use for treatment of cancer, are also disclosed.

### FIELD OF THE INVENTION

The present invention is in the field of immunology and specifically, relates to immune-conjugates, bi-specific constructs and cancer immunotherapy.

### BACKGROUND OF THE INVENTION

Worldwide, head and neck cell carcinoma (HNSCC) and esophageal squamous cell carcinoma (ESCC) have an annual incidence rate of approximately 700,000 and 500,000 new cases, respectively. The standard of care for most cases of HNSCC and ESCC includes mainly surgical intervention, radiation, and chemotherapy, while for recurrent or metastatic HNSCC patients, targeted therapy and immunotherapy have also been approved. Despite significant efforts to improve these malignancies' treatment, their 5-year survival rate remains 50% for HNSCC and 20% for ESCC.

This resistance phenotype is associated, among other mechanisms, with overexpression of the tyrosine kinases receptor AXL, located on the tumor cells' surface. AXL protein belongs to the TYRO3-AXL-MERTK (TAM) family of receptor tyrosine kinases (RTKs). AXL activation leads to several downstream signaling pathways, including the MAPK, PI3K/AKT/mTOR (Verma et al. 2011. Molecular cancer therapeutics, 10(10), 1763-1773), JAK/STAT, and NF-κB pathways (Varnum et al. 1996. Mol. Cell. Biol. 1996, 16, 135-145), which play an essential role in tumor cell survival, migration, invasion, and drug resistance in multiple cancer types (Dent. 2014. Cancer Biol. Ther. 15, 245-246).

AXL expression was shown to be altered in case of malignancy. Overexpression of AXL is associated with increased tumor proliferation, migration, invasion, angiogenesis, stem-cell maintenance, and epithelial-mesenchymal transition (EMT), which, consequently, induce metastasis, drug resistance, and immune suppression (Alfieri et al. 2020. Cancers (Basel), 12; Asiedu et al. 2014. Oncogene, 33, 1316-1324; Jimbo et al. 2019. Oncotarget, 10, 5152-5167; Rankin et al. 2010. Cancer Res. 70, 7570-7579; Saab et al. 2019. Am. J. Cancer Res, 9, 2719; Tanaka et al. 2019. Oncotarget, 10, 2887-2898). Downregulation of AXL reduces motility, metastasis, and invasion of tumor cells (Gjerdrum et al. 2010. Proc. Natl. Acad. Sci. U.S.A, 107, 1124-1129). As so, AXL emerges as a potential prognostic biomarker and a promising therapeutic target.

AXL expression in patients with HNSCC has showed significant correlation with a higher pathologic grade, the presence of lymph node metastasis and distant metastases, and a shorter relapse-free survival time (Brand et al. 2015. Clin. Cancer Res., 21, 2601-2612; von Massenhausen et al. 2017. Int. J. Mol. Sci., 18). This correlation between AXL expression and metastasis, together with the fact that metastatic cells in HNSCC are genetically identical to the primary tumors (Hedberg et al. 2016. J. Clin. Invest., 126, 169-180), further supports the hypothesis that AXL acts as a driver protein that regulates metastasis. In addition to the putative role of AXL in tumor cell proliferation and invasion, AXL also induces immune suppression and inhibits signaling pathways that, otherwise, activate dendritic cells, natural killer cells, and macrophages. AXL-induced inhibition of these pathways attenuates these cells' ability to eliminate metastases (Gay et al. 2017. Br. J. Cancer, 116, 415-423). AXL also plays a role in modulating the immunological responses and mediating cancer cell immune escape by inhibiting inflammatory signaling, inflammatory cytokine secretion, and T-cell activation inhibition (Aguilera et al. 2017. Clin. Cancer Res., 23, 2928-2933).

AXL's therapy resistance to various cancer treatments such as molecular targeted therapy, radiation and immune therapy is attributed to AXL overexpression. In HNSCC, AXL overexpression correlates with radiotherapy resistance, upregulation of PD-L1, and low CD8⁺ tumor-infiltrating lymphocytes (TILs).

The efficiency of AXL's repression, either by pharmacological agents or genetic modification, was extensively examined (Brand et al. 2015. Clin. Cancer Res., 21, 2601-2612; Lin et al. 2017. Oncotarget, 8, 41064-41077). Specifically, knockdown of AXL in HNSCC cells has showed to enable re-sensitization of the cells to chemotherapy and radiation (Skinner et al. 2017. Clin. Cancer Res., 23, 2713-2722). In addition, inhibition of AXL displayed on HNSCC and ESCC cells successfully eliminated resistance to radiation and PI3Ka therapy, respectively (Badarni et al. 2019. JCI Insight, 4; McDaniel et al. 2020. Clin. Cancer Res., 26, 4349-4359).

Various agents targeting AXL have been developed, including small molecule inhibitors (i.e BGB324/R428 and TP-0903), anti-AXL mAbs (i.e. AVB-S6-500 and CAB-AXL-ADC), nucleotide aptamers, soluble receptors, and several natural compounds (Gay et al. 2017. Br. J. Cancer, 116, 415-423; Zhu et al. 2019. Mol. Cancer, 18, 1-22). Over twenty clinical trials are ongoing to test AXL inhibitors as single agents and in combination with other drugs but not even a single one has yet been approved.

AXL is a key protein leading to tumor resistance to currently used therapeutic agents. It was reported that targeting AXL enhanced the effect of VEGF, EGFR, PI3K, PARP, and HER2 inhibitors as well as chemotherapy (Ye et al. 2010. Oncogene, 29, 5254-5264). In addition, AXL overexpression on tumor cells demonstrated limited anti-Programmed Cell Death Protein 1 (PD-1) therapy due to its involvement in the immunosuppressive tumor microenvironment (TME) and inhibition of T-cell activation (Guo et al. 2017. Oncotarget, 8, 89761-89774; Hugo et al. 2016. Cell, 165, 35-44).

Cancer immunotherapy is one of the most evolving subjects of our days, accompanied by promising results. Immune checkpoint inhibitors are being developed and approved for varied cancer types, including the anti-PD-1 agents. This inhibitory receptor is broadly expressed on all T cells during activation (Sharpe et al. 2018. Nat. Rev. Immunol., 18, 153-167).

PD-1 plays a pivotal role in inhibiting and regulating the immune responses and several blocking antibodies against PD-1 have been approved for melanoma, lymphoma, lung cancer, renal cell cancer (RCC), bladder, liver, gastroesophageal cancer and HNSCC (Han et al. 2020. Am. J. Cancer Res., 10, 727-742). Accumulated clinical data show that only 5-30% of patients respond to anti-PD-1 therapy, while the vast majority of patients do not benefit from this treatment (Carretero-Gonzilez et al. 2018. Oncotarget, 9, 8706-8715; Sun et al. 2020. Sci. Rep., 10, 1-13).

The association of AXL and PD-L1 was validated in clinical samples and patients and was linked to failure to respond to radiotherapy treatment. Guo et al. showed that AXL inhibition reduced PD-L1 expression on tumor cells, increased CD8⁺ and CD4⁺ TILs, and synergistically increased the anti-PD-1 efficacy in murine models of ovarian or breast cancer (Oncotarget. 2017; 8(52):89761-89774. doi:10.18632/oncotarget.21125). These results suggest that combination therapy for targeting both PD-1 and AXL may have an efficient anti-tumor effect. Indeed several clinical trials aiming to test the efficacy of an AXL inhibitor in combination with the anti-PD-I therapy have been initiated, two of them are ongoing, but preliminary encouraging results were published for at least one (Felip et al., Journal of Clinical Oncology 37, no. 15_suppl (May 20, 2019) 9098-9098).

Bi-specific antibodies (BsAbs) combine specificities of two antibodies and simultaneously address different antigens or epitopes, thus allowing the dual-inhibition of disease-promoting target proteins. The concept behind BsAbs came from the understanding that cancer and other diseases are multifactorial and simultaneous blocking of several targets may improve treatment efficacy. Various therapeutic routes are doable due to the dual specificity when using BsAbs. For example, recruiting and redirecting T cells into tumor niche or microenvironment (TME), blocking two different ligands, or cross-linking two receptors and more.

Usually, BsAbs are broadly classified into IgG-like and non-IgG-like molecules. IgG-like BsAbs are bigger, similar to a conventional antibody, having longer serum half-lives, while non-IgG-like are usually smaller, having enhanced tissue penetration capability but a shorter half-life. A variety of non-IgG-like formats are being developed, mainly based on the single-chain variable (scFv) fragments. In scFv, only the variable regions of the heavy (VH) and light chains (VL) are being used, connected to each other, as the primary element for antigen binding. A short linker peptide, rich in glycine for flexibility and serine for solubility, is usually used to connect the VH and the VL chains. This protein retains the original immunoglobulin specificity, despite removing the constant regions and introduction of the linker. To overcome the shorter half-life of non-IgG-like BsAbs, the molecule can be fused to an Fc fragment, albumin, or another carrier molecule to increase its half-time. BsAbs generated by a fusion of two inhibitory scFvs domains can be used to recruit T cells into the TME, as was shown by Krishnamurthy et al., for Catumaxomab, an FDA approved EpCAM/CD3 BsAb for the treatment of malignant ascites in adults with EpCAM-positive carcinomas (Pharmacol Ther. 2018; 185:122-134).

WO2015/063187 discloses multivalent IgG-like antigen-binding proteins comprising two heavy and two light chains composed of a specific arrangement of variable and constant domains, for the treatment of immunological and inflammatory disorders, and cancer. The publication suggests, but has not produced, a multivalent bispecific antibody capable of specific binding antigen pairs, inter alia, AXL and PD-1, present on the same or different cells.

A tetravalent bispecific antibody combining PD-L1 and AXL targeting that retains the properties of the parental antibodies and demonstrates enhanced activity in immune activation assays has been disclosed by Celldex Therapeutics, Inc. (https://www.globenewswire.com/news-release/2020/11/09/2122752/24180/en/Celldex-Presents-Data-from-Oncology-Portfolio-at-SITC-2020.html).

There is an unmet need to develop an efficient inhibitor for simultaneous targeting of AXL and PD-1 for the treatment of HNSCC and ESCC, in particular for the treatment of patients with tumors or metastases that express a high level of AXL.

While previous publications and clinical trials disclosed the development and use of bi-specific antibodies for cancer therapy, a bi-specific construct of linked scFv antibodies that binds AXL and PD-1 has not been made or investigated.

### SUMMARY OF THE INVENTION

The present invention provides inhibitory bi-specific molecules or constructs comprising two different single-chain variable fragments (scFv), one targeted against the receptor AXL and one against programmed cell death protein 1 (PD-1). These bi-specific antibody-like inhibitory constructs (BsAbI-AXL/PD-1) are shown herein to simultaneously target AXL and PD-1 on cancer and/or immune cells and are therefore disclosed for treatment of cancer, in particular for the treatment of head and neck carcinoma (HNSCC) and esophageal squamous cell carcinoma (ESCC).

The present invention is based in part on the unexpected discovery that apart from the binding to AXL expressed on tumor cells and PD-1 expressed on CD8⁺ T cells, the BsAbl-AXL/PD-1 also inhibits the interaction between PD-1 and its ligand PD-L1. The invention is further based, in part, on the unexpected discovery that the simultaneous targeting of the AXL displayed on tumor cells and PD-1 displayed on T cells, by BsAbl-AXL/PD-1, leads to increased killing of cancer cells by naïve human peripheral blood mononuclear cells (PBMCs). Moreover, the enhanced anti-tumor activity of BsAbl-AXL/PD-1, represented by the enhanced anti-tumor lytic activity of CD8⁺ T cells, was found to be superior to the respective mono-specific antibodies.

Some of the bi-specific constructs of the present invention display improved inhibition of PD-1 binding to its ligand, PD-L1, even compared to the anti PD-1 antibody Keytruda that is currently in clinical use.

Furthermore, the Bi-3 construct of the present invention inhibits tumor growth in vivo in a squamous cell carcinoma mouse model, indicating its potential therapeutic effect in human subjects.

The present invention provides according to an aspect, a bi-specific construct comprising two different single-chain variable fragments (scFv), one targeted against the receptor AXL and one against programmed cell death protein 1 (PD-1).

Any antibody, antibody fragment or antigen-binding site, targeted against AXL or PD-1, may be used to produce the scFv molecules of the bi-specific antigen binding molecules of the present invention. This includes commercial antibodies against AXL (e.g., Enapotamab and YW327.6S2), and PD-1 (e.g., Keytruda and Nivolumab), as well as newly designed binding molecules against these targets.

According to some embodiments, the bi-specific construct comprises two scFv molecules targeted to the human receptor AXL and two scFv molecules targeted to human PD1.

According to some embodiments, the bi-specific construct comprises two polypeptides, each polypeptide comprising two different scFvs and a carrier polypeptide that form a structure selected from scFv(PD-1)-carrier-scFv(AXL) and scFv(AXL)-carrier-scFv(PD-1).

According to some embodiments, at least one of the scFv molecules of the bi-specific construct comprises at least one linker or spacer of 1-50 amino acid residues, connecting the heavy chain variable region (VH) and the light chain variable region (VL) and/or connected to at least one terminal of the scFv amnio acid sequence. The linker or spacer may be identical or different within and for each scFv molecule in the bi-specific antigen-binding construct. A linker or spacer may be also used to connect the different scFv molecules to each other and/or to a carrier molecule. Each option represents a separate embodiment of the present invention.

According to some embodiments, the linker consists of 2-40, 3-30 or 4-20 amino acid residues. According to other embodiments, the linker comprises the amino acid residues Glycine (Gly, G) and Serine (Ser, S). According to yet other embodiments, the linker comprises the sequence GGGGS (SEQ ID NO: 21). According to other embodiments, the linker comprises 2-8 consecutive repeats of the sequence GGGGS. According to yet other embodiments, the linker comprises 4 consecutive repeats of the sequence GGGGS. Each option represents a separate embodiment of the present invention.

According to some embodiments, an scFv of a construct according to the present invention, comprises the linker GGGGS that connected the VL and the VH regions. According to other embodiments, an scFv of a construct according to the present invention, comprises the linker GGGGS in at least one of the scFv sequence terminals. According to some specific embodiments of the present invention, the construct comprises at least one scFv of the structure VL-(GGGGS)₄-VH-(GGGGS)₄. Each option represents a separate embodiment of the present invention.

According to some embodiments, the anti-AXL scFv molecule comprises the following set of six complementarity-determining regions (CDRs) sequences:

| CDR | Sequence | SEQ ID NO: |
|---|---|---|
| CDR-H1 | GSWIH | 1 |
| CDR-H2 | WINPYRGYAYYADSVKG | 2 |
| CDR-H3 | EYSGWGGSSVGYAMDY | 3 |
| CDR-L1 | RASQDVSTA | 4 |
| CDR-L2 | SASFLYS | 5 |
| CDR-L3 | QQSYTTPPT | 6 |

There are several methods known in the art for determining the CDR sequences of a given antibody molecule, but there is no standard unequivocal method. Determination of CDR sequences from antibody heavy and light chain variable regions can be made according to any method known in the art, including, but not limited to, the methods known as KABAT, Chothia, and IMGT. A selected set of CDRs may include sequences identified by more than one method, namely, some CDR sequences may be determined using KABAT and some using IMGT, for example. According to some embodiments, the CDR sequences of the mAb variable regions are determined using the KABAT.

According to some embodiments, the bi-specific antigen-binding construct comprises an scFv against AXL, comprising the sequence: DIQMTQSPSSLSASVGDRVTITCRASQDVSTAVAWYQQKPGKAPKLLIYSASFLYSGVPS RFSGSGSGTDFTLTISSLQPEDFATYYCQQSYTTPPTFGQGTKVEIKGGGGSGGGGSGGG GSGGGGSEVQLVESGGGLVQPGGSLRLSCAASGFSLSGSWIHWVRQAPGKGLEWVGWI NPYRGYAYYADSVKGRFTISADTSKNTAYLQMNSLRAEDTAVYYCAREYSGWGGSSV GYAMDYWGQGTLVGGGGSGGGGSGGGGSGGGGS (SEQ ID NO: 7), or an analog or derivative having at least 90% identity with said sequence.

According to some embodiments, the anti-PD-1 scFv molecule comprises the following set of six CDRs sequences:

| CDR | Sequence | SEQ ID NO: |
|---|---|---|
| CDR-H1 | INSGMH | 8 |
| CDR-H2 | VIWYDGSKRYYADSVKG | 9 |
| CDR-H3 | NDDY | 10 |
| CDR-L1 | RASQSVSSYLA | 11 |
| CDR-L2 | DASNRAT | 12 |
| CDR-L3 | QQSSNWPRT | 13 |

According to some embodiments, the bi-specific antigen-binding construct comprises an scFv against PD-1, comprising the sequence: EIVLTQSPATLSLSPGERATLSCRASQSVSSYLAWYQQKPGQAPRLLIYDASNRATGIPA RFSGSGSGTDFTLTISSLEPEDFAVYYCQQSSNWPRTFGQGTKVEIKGGGGSGGGGSGG GGSGGGGSQVQLVESGGGVVQPGRSLRLDCKASGITFSNSGMHWVRQAPGKGLEWVA VIWYDGSKRYYADSVKGRFTISRDNSKNTLFLQMNSLRAEDTAVYYCATNDDYWGQG TLVTVSSGGGGSGGGGSGGGGSGGGGS (SEQ ID NO: 14), or an analog or derivative having at least 90% identity with said sequence.

According to some embodiments, the bi-specific antigen binding construct comprises an scFv against PD-1, comprising the sequence: EIVLTQSPATLSLSPGERATL SCRASQSVSSYLAWYQQKPGQAPRLLIYDASNRATGIPA RFSGSGSGTDFTLTISSLEPEDFAVYYCQQSSNWPRTFGQGTKVEIKGGGGSGGGGSGG GGSGGGGSQVQLVESGGGVVQPGRSLRLDCKASGITFSNSGMHWVRQAPGKGLEWVA VIWYDGSKRYYADSVKGRFTISRDNSKNTLFLQMNSLRAEDTAVYYCATNDDYWGQG TLVTVSSGGGGSGGGGSGGGGSGGGGS (SEQ ID NO: 14) or an analog or derivative having at least 90% identity with said sequence; and an scFv against AXL, comprising the sequence: DIQMTQSPSSLSASVGDRVTITCRASQDVSTAVAWYQQKPGKAPKLLIYSASFLYSGVPS RFSGSGSGTDFTLTISSLQPEDFATYYCQQSYTTPPTFGQGTKVEIKGGGGSGGGGSGGG GSGGGGSEVQLVESGGGLVQPGGSLRLSCAASGFSLSGSWIHWVRQAPGKGLEWVGWI NPYRGYAYYADSVKGRFTISADTSKNTAYLQMNSLRAEDTAVYYCAREYSGWGGSSV GYAMDYWGQGTLVGGGGSGGGGSGGGGSGGGGS (SEQ ID NO: 7) or an analog or derivative having at least 90% identity with said sequence.

According to some embodiments, the bi-specific AXL/PD-1 inhibitory construct of the present invention comprises at least one carrier molecule. According to some embodiments, the carrier molecule comprises a polypeptide sequence.

According to some embodiments, the carrier polypeptide sequence connects the two scFv molecules to form the structure scFv-carrier-scFv. According to some specific embodiments, the construct is selected from scFv(AXL)-carrier-scFv(PD-1); and scFv(PD-1)-carrier-scFv(AXL), wherein the scFv molecule on the left is the N-terminal one. Each option represents a separate embodiment of the present invention.

According to other embodiments, the carrier polypeptide sequence is connected to the terminal of the scFv-scFv molecules to form a construct selected from the group consisting of: carrier-scFv(AXL)-scFv(PD-1); scFv(AXL)-scFv(PD-1)-carrier; carrier-scFv(PD-1)-scFv(AXL); scFv(PD-1)-scFv(AXL)-carrier. Each option represents a separate embodiment of the present invention.

According to some embodiments, the carrier polypeptide is an immunoglobulin molecule or a fragment thereof.

According to some embodiments, the carrier polypeptide sequence comprises a human IgG constant domain (hereinafter Fc) or a fragment thereof.

According to some embodiments, the human IgG is a human IgG1 or a fragment thereof comprising the CH2 and CH3 domains of the IgG heavy chain and the hinge region.

According to some embodiments, the human IgG1 FC domain is encoded by a polynucleotide sequence comprising SEQ ID NO: 15, or by an analog or derivative having at least 90% identity with said sequence.

According to some embodiments, the bi-specific construct comprises two polypeptides, each polypeptide comprising two different scFvs and a carrier polypeptide, wherein the bi-specific construct forms a structure selected from [scFv(PD-1)]2-IgG1Fc-[scFv(AXL)]2 and [scFv(AXL)]2-IgG1Fc-[scFv(PD-1)]2. According to some embodiments, the PD1-AXL-Fc construct (anti PD1 scFv-anti AXL scFv-Fc) comprises the amino acid sequence:

EIVLTQSPATLSLSPGERATLSCRASQSVSSYLAWYQQKPGQAPRLLIYDASNRA TGIPARFSGSGSGTDFTTISSLEPEDFAVYYCQQSSNWPRTFGQGTKVEIKGGGGSGGGG SGGGGSGGGGSQVQLVESGGGVVQPGRSLRLDCKASGITFSNSGMHWVRQAPGKGLE WVAVIWYDGSKRYYADSVKGRFTISRDNSKNTLFLQMNSLRAEDTAVYYCATNDDYW GQGTLVTVSSGGGGSGGGGSGGGGSGGGGSDIQMTQSPSSLSASVGDRVTITCRASQDV STAVAWYQQKPGKAPKLLIYSASFLYSGVPSRFSGSGSGTDFTLTISSLQPEDFATYYCQ QSYTTPPTFGQGTKVEIKGGGGSGGGGSGGGGSGGGGSEVQLVESGGGLVQPGGSLRLS CAASGFSLSGSWIHWVRQAPGKGLEWVGWINPYRGYAYYADSVKGRFTISADTSKNTA YLQMNSLRAEDTAVYYCAREYSGWGGSSVGYAMDYWGQGTLVGGGGSGGGGSGGG GSGGGGSRSVECPPCPAPPVAGPSVFLFPPKPKDTLMISRTPEVTCVVVDVSSHEDPEVKF NWYVDGVEVHNAKTKPREEQYNSTYRVVSVLTVLHQDWLNGKEYKCKVSNKGLPSSI EKTISKAKGQPREPQVYTLPPSREEMTKNQVSLTCLVKGFYPSDIAVEWESNGQPENNY KTTPPVLDSDGSFFLYSKLTVDKSRWQQGNVFSCSVMHEGLHNHYTQKSLSLSPGK (SEQ ID NO: 22), or an analog or derivative having at least 90% identity with said sequence.

According to some embodiments, the AXL-PD1-Fc construct (-anti-AXL scFv-anti PD1 scFv-Fc) comprises the amino acid sequence:

DIQMTQSPSSLSASVGDRVTITCRASQDVSTAVAWYQQKPGKAPKLLIYSASFLY SGVPSRFSGSGSGTDFTLTISSLQPEDFATYYCQQSYTTPPTFGQGTKVEIKGGGGSGGG GSGGGGSGGGGSEVQLVESGGGLVQPGGSLRLSCAASGFSLSGSWIHWVRQAPGKGLE WVGWINPYRGYAYYADSVKGRFTISADTSKNTAYLQMNSLRAEDTAVYYCAREYSGW GGSSVGYAMDYWGQGTLVGGGGSGGGGSGGGGSGGGGSEIVLTQSPATLSLSPGERAT LSCRASQSVSSYLAWYQQKPGQAPRLLIYDASNRATGIPARFSGSGSGTDFTLTISSLEPE DFAVYYCQQSSNWPRTFGQGTKVEIKGGGGSGGGGSGGGGSGGGGSQVQLVESGGGV VQPGRSLRLDCKASGITFSNSGMHWVRQAPGKGLEWVAVIWYDGSKRYYADSVKGRF TISRDNSKNTLFLQMNSLRAEDTAVYYCATNDDYWGQGTLVTVSSGGGGSGGGGSGG GGSGGGGSRSVECPPCPAPPVAGPSVFLFPPKPKDTLMISRTPEVTCVVVDVSSHEDPEVK FNWYVDGVEVHNAKTKPREEQYNSTYRVVSVLTVLHQDWLNGKEYKCKVSNKGLPSS IEKTISKAKGQPREPQVYTLPPSREEMTKNQVSLTCLVKGFYPSDIAVEWESNGQPENNY KTTPPVLDSDGSFFLYSKLTVDKSRWQQGNVFSCSVMHEGLHNHYTQKSLSLSPGK (SEQ ID NO: 23), or an analog or derivative having at least 90% identity with said sequence.

According to some embodiments, the PD1-Fc-AXL construct (anti PD1 scFv-Fc-anti AXL scFv) comprises the amino acid sequence:

EIVLTQSPATLSLSPGERATLSCRASQSVSSYLAWYQQKPGQAPRLLIYDASNRA TGIPARFSGSGSGTDFTLTISSLEPEDFAVYYCQQSSNWPRTFGQGTKVEIKGGGGSGGG GSGGGGSGGGGSQVQLVESGGGVVQPGRSLRLDCKASGITFSNSGMHWVRQAPGKGL EWVAVIWYDGSKRYYADSVKGRFTISRDNSKNTLFLQMNSLRAEDTAVYYCATNDDY WGQGTLVTVSSGGGGSGGGGSGGGGSGGGGSRSVECPPCPAPPVAGPSVFLFPPKPKDT LMISRTPEVTCVVVDVSHEDPEVKFNWYVDGVEVHNAKTKPREEQYNSTYRVVSVLTV LHQDWLNGKEYKCKVSNKGLPSSIEKTISKAKGQPREPQVYTLPPSREEMTKNQVSLTC LVKGFYPSDIAVEWESNGQPENNYKTTPPVLDSDGSFFLYSKLTVDKSRWQQGNVFSCS VMHEGLHNHYTQKSLSLSPGKGGGGSGGGGSGGGGSGGGGSDIQMTQSPSSLSASVGD RVTITCRASQDVSTAVAWYQQKPGKAPKLLIYSASFLYSGVPSRFSGSGSGTDFTLTISSL QPEDFATYYCQQSYTTPPTFGQGTKVEIKGGGGSGGGGSGGGGSGGGGSEVQLVESGG GLVQPGGSLRLSCAASGFSLSGSWIHWVRQAPGKGLEWVGWINPYRGYAYYADSVKG RFTISADTSKNTAYLQMNSLRAEDTAVYYCAREYSGWGGSSVGYAMDYWGQGTLV (SEQ ID NO: 24), or an analog or derivative having at least 90% identity with said sequence.

According to some embodiments, the PD1-AXL-Fc construct (anti PD1 scFv-anti AXL scFv-Fc, denoted Bi-1) comprises the amino acid sequence:

EIVLTQSPATLSLSPGERATLSCRASQSVSSYLAWYQQKPGQAPRLLIYDASNRA TGIPARFSGSGSGTDFTTISSLEPEDFAVYYCQQSSNWPRTFGQGTKVEIKGGGGSGGGG SGGGGSGGGGSQVQLVESGGGVVQPGRSLRLDCKASGITFSNSGMHWVRQAPGKGLE WVAVIWYDGSKRYYADSVKGRFTISRDNSKNTLFLQMNSLRAEDTAVYYCATNDDYW GQGTLVTVSSGGGGSGGGGSGGGGSGGGGSDIQMTQSPSSLSASVGDRVTITCRASQDV STAVAWYQQKPGKAPKLLIYSASFLYSGVPSRFSGSGSGTDFTLTISSLQPEDFATYYCQ QSYTTPPTFGQGTKVEIKGGGGSGGGGSGGGGSGGGGSEVQLVESGGGLVQPGGSLRLS CAASGFSLSGSWIHWVRQAPGKGLEWVGWINPYRGYAYYADSVKGRFTISADTSKNTA YLQMNSLRAEDTAVYYCAREYSGWGGSSVGYAMDYWGQGTLVGGGGSGGGGSGGG GSGGGGSRSVECPPCPAPPVAGPSVFLFPPKPKDTLMISRTPEVTCVVVDVSSHEDPEVKF NWYVDGVEVHNAKTKPREEQYNSTYRVVSVLTVLHQDWLNGKEYKCKVSNKGLPSSI EKTISKAKGQPREPQVYTLPPSREEMTKNQVSLTCLVKGFYPSDIAVEWESNGQPENNY KTTPPVLDSDGSFFLYSKLTVDKSRWQQGNVFSCSVMHEGLHNHYTQKSLSLSPGKHH IHHHH (SEQ ID NO: 25), or an analog or derivative having at least 90% identity with said sequence.

According to some embodiments, the AXL-PD1-Fc construct (-anti-AXL scFv-anti PD1 scFv-Fc, denoted Bi-2) comprises the amino acid sequence:

DIQMTQSPSSLSASVGDRVTITCRASQDVSTAVAWYQQKPGKAPKLLIYSASFLY SGVPSRFSGSGSGTDFTLTISSLQPEDFATYYCQQSYTTPPTFGQGTKVEIKGGGGSGGG GSGGGGSGGGGSEVQLVESGGGLVQPGGSLRLSCAASGFSLSGSWIHWVRQAPGKGLE WVGWINPYRGYAYYADSVKGRFTISADTSKNTAYLQMNSLRAEDTAVYYCAREYSGW GGSSVGYAMDYWGQGTLVGGGGSGGGGSGGGGSGGGGSEIVLTQSPATLSLSPGERAT LSCRASQSVSSYLAWYQQKPGQAPRLLIYDASNRATGIPARFSGSGSGTDFTLTISSLEPE DFAVYYCQQSSNWPRTFGQGTKVEIKGGGGSGGGGSGGGGSGGGGSQVQLVESGGGV VQPGRSLRLDCKASGITFSNSGMHWVRQAPGKGLEWVAVIWYDGSKRYYADSVKGRF TISRDNSKNTLFLQMNSLRAEDTAVYYCATNDDYWGQGTLVTVSSGGGGSGGGGSGG GGSGGGGSRSVECPPCPAPPVAGPSVFLFPPKPKDTLMISRTPEVTCVVVDVSSHEDPEVK FNWYVDGVEVHNAKTKPREEQYNSTYRVVSVLTVLHQDWLNGKEYKCKVSNKGLPSS IEKTISKAKGQPREPQVYTLPPSREEMTKNQVSLTCLVKGFYPSDIAVEWESNGQPENNY KTTPPVLDSDGSFFLYSKLTVDKSRWQQGNVFSCSVMHEGLHNHYTQKSLSLSPGKHH HHHHH (SEQ ID NO: 26), or an analog or derivative having at least 90% identity with said sequence.

According to some embodiments, the PD1-Fc-AXL construct (anti PD1 scFv-Fc-anti AXL scFv, denoted Bi-3) comprises the amino acid sequence:

EIVLTQSPATLSLSPGERATLSCRASQSVSSYLAWYQQKPGQAPRLLIYDASNRA TGIPARFSGSGSGTDFTLTISSLEPEDFAVYYCQQSSNWPRTFGQGTKVEIKGGGGSGGG GSGGGGSGGGGSQVQLVESGGGVVQPGRSLRLDCKASGITFSNSGMHWVRQAPGKGL EWVAVIWYDGSKRYYADSVKGRFTISRDNSKNTLFLQMNSLRAEDTAVYYCATNDDY WGQGTLVTVSSGGGGSGGGGSGGGGSGGGGSRSVECPPCPAPPVAGPSVFLFPPKPKDT LMISRTPEVTCVVVDVSHEDPEVKFNWYVDGVEVHNAKTKPREEQYNSTYRVVSVLTV LHQDWLNGKEYKCKVSNKGLPSSIEKTISKAKGQPREPQVYTLPPSREEMTKNQVSLTC LVKGFYPSDIAVEWESNGQPENNYKTTPPVLDSDGSFFLYSKLTVDKSRWQQGNVFSCS VMHEGLHNHYTQKSLSLSPGKGGGGSGGGGSGGGGSGGGGSDIQMTQSPSSLSASVGD RVTITCRASQDVSTAVAWYQQKPGKAPKLLIYSASFLYSGVPSRFSGSGSGTDFTLTISSL QPEDFATYYCQQSYTTPPTFGQGTKVEIKGGGGSGGGGSGGGGSGGGGSEVQLVESGG GLVQPGGSLRLSCAASGFSLSGSWIHWVRQAPGKGLEWVGWINPYRGYAYYADSVKG RFTISADTSKNTAYLQMNSLRAEDTAVYYCAREYSGWGGSSVGYAMDYWGQGTLVH HHHHH (SEQ ID NO: 27), or an analog or derivative having at least 90% identity with said sequence.

According to other embodiments, the carrier polypeptide is human albumin or a fragment thereof.

According to yet other embodiments, the carrier is a non-peptidic molecule. According to yet other embodiments, the carrier comprises a polymer, e.g., a polyethylene glycol (PEG).

According to some embodiments, a bi-specific construct according to the present invention comprises at least one PEG molecule. According to some specific embodiments, the bi-specific construct comprises a plurality of PEG molecules.

Variants of the bi-specific constructs of the present invention are also included, as long as they retain binding to both human AXL and human PD1. According to some embodiments, a variant having 80, 85, 90, 95, 98 or 99% amino acid identity to any of the constructs provided herein is included in the scope of the present invention.

Also provided according to yet another aspect of the present invention, are isolated nucleic acids encoding the bi-specific constructs disclosed herein.

According to some embodiments, the nucleic acid sequence that encodes the anti-AXL scFv (VL-(GGGGS)₄-VH-(GGGGS)₄) comprises SEQ ID NO: 16, or an analog or derivative having at least 80% identity with said sequence.

According to some embodiments, the nucleic acid sequence that encodes the anti-PD1 scFv (VL-(GGGGS)₄-VH-(GGGGS)₄) comprises SEQ ID NO: 17, or an analog or derivative having at least 80% identity with said sequence.

According to some embodiments, the nucleic acid sequence that encodes the bi-specific construct denoted bi-1 (PD1-AXL-Fc, wherein AXL is anti-AXL scFv, PD1 is anti PD1 scFv and Fc is an engineered human IgG region) comprises SEQ ID NO: 18, or an analog or derivative having at least 80% identity with said sequence.

According to some embodiments, the nucleic acid sequence that encodes the bi-specific construct denoted bi-2 (AXL-PD1-Fc, wherein AXL is anti-AXL scFv, PD1 is anti PD1 scFv and Fc is an engineered human IgG region) comprises SEQ ID NO: 19, or an analog or derivative having at least 80% identity with said sequence.

According to some embodiments, the nucleic acid sequence that encodes the bi-specific construct denoted bi-3 (PD1-Fc-AXL, wherein AXL is anti-AXL scFv, PD1 is anti PD1 scFv and Fc is an engineered human IgG region) comprises SEQ ID NO: 20, or an analog or derivative having at least 80% identity with said sequence.

Variants of the nucleotide sequences encoding the bi-specific constructs of the present invention are also included. According to some embodiments, a variant having 70, 75, 80, 85, 90, 95, 98 or 99% identity to any of the nucleotide sequences provided herein is included in the scope of the present invention.

Also provided are vectors comprising the nucleic acid sequences encoding the bi-specific constructs disclosed herein, as well as host cells comprising the vectors comprising the isolated nucleic acids disclosed herein.

Pharmaceutical compositions comprising at least one bi-specific inhibitory construct (BsAbI-AXL/PD-1) as disclosed herein, and a pharmaceutically acceptable excipient, diluent, salt or buffer are also provided according to another aspect of the present invention.

These pharmaceutical compositions may be formulated, using methods well known in the art, for any administration mode, including but not limited parenteral administration.

The present invention also provides, bi-specific antigen-binding constructs comprising two different scFv molecules, one targeted against the receptor AXL and one against PD1, and pharmaceutical compositions comprising these constructs, for preventing, attenuating or treating cancer.

According to some embodiments, the bi-specific constructs are for increasing the duration of survival of a subject having cancer. According to some embodiments, the bi-specific constructs are for increasing the progression-free survival of a subject having cancer. According to some embodiments, the bi-specific constructs are for increasing the response incidence in a group of subjects. According to yet other embodiments, the bi-specific constructs are for increasing the duration of response of a subject having cancer. According to some embodiments, the bi-specific constructs are for preventing or inhibiting the development of metastasis in a patient having cancer. According to some embodiments, the bi-specific constructs are for preventing tumor recurrence.

According to some embodiments, the bi-specific AXL/PD1 antigen-binding constructs of the present invention are for the treatment of cancer having a local-recurrent disease or metastatic disease that express a high level of AXL.

According to some embodiments, the bi-specific AXL/PD1 antigen-binding constructs of the present invention are for the treatment of head and neck cell carcinoma (HNSCC) and esophageal squamous cell carcinoma (ESCC).

Methods for inhibiting the growth or proliferation of cancer cells or for promoting T cell-mediated killing of the cancer cells are also provided. The methods comprise contacting the cancer cells with the bi-specific constructs of the present invention.

The present invention provides, according to yet another aspect a method of preventing, attenuating or treating cancer by administering to a subject in need thereof, a bi-specific antigen binding molecule comprising two different single-chain variable fragments (scFv), one targeted against the receptor AXL and one against programmed cell death protein 1 (PD1).

According to some embodiments, the treatment increases the duration of survival of a subject having cancer. According to some embodiments, the treatment increases the progression-free survival of a subject having cancer. According to some embodiments, the treatment increases the response incidence in a group of subjects. According to yet other embodiments, the treatment increases the duration of response of a subject having cancer. According to some embodiments, the treatment prevents or inhibits the development of metastasis in a patient having cancer. According to some embodiments, the treatment prevents tumor recurrence.

The method of preventing, attenuating or treating cancer includes preventing the creation or spread or treating tumor metastasis.

The pharmaceutical composition according to the present invention may be administered as a stand-alone treatment or in combination with any other anti-cancer treatment, agent or composition. According to a specific embodiment, bi-specific constructs according to the present invention are administered to a subject in need thereof as part of a treatment regimen in conjunction with at least one anti-cancer composition or therapy. The pharmaceutical composition according to the present invention may be administered together with the anti-cancer agent or separately.

According to a specific embodiment, the anti-cancer composition comprises at least one chemotherapeutic agent. The chemotherapeutic agent, which could be administered separately or together with the constructs of the present invention, may comprise any such agent known in the art exhibiting anti-cancer activity. Biological therapies, for example using antibodies, T-cells, CAR-T cells and genetic manipulations are also included within the scope of an anti-cancer treatment, as well as radiotherapy and surgery.

Also provided, according to the present invention, are methods of producing the bi-specific constructs disclosed herein and pharmaceutical compositions comprising them. The methods comprise culturing a cell comprising a nucleic acid encoding the bi-specific polypeptide construct, and recovering the construct polypeptide from the cell or culture.

According to yet another aspect, the present invention provides kits comprising bi-specific constructs disclosed herein and packaging for the same.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The novel features described herein are set forth with particularity in the appended claims. A better understanding of the characteristics and advantages of the features described herein will be obtained by reference to the following detailed description that sets forth illustrative examples, in which the principles of the features described herein are utilized, and the accompanying drawings of which:

FIG. 1A-1B are schematic presentations of exemplary constructs prepared and tested in the present invention and their proposed action. FIG. 1A shows two mono scFv conjugates and three BsAbl-AXL/PD-1 constructs comprising anti AXL and/or PD-1 scFv. FIG. 1B demonstrates a suggested mechanism in which BsAbl-AXL/PD-1 increases the interaction between the tumor cell and surrounding T cells. Simultaneous binding of the BsAbI-AXL/PD-1 to tumor cells and T cells results in increased T cell anti-tumor lytic activity in addition to AXL and PD-1 blockade.

FIGS. 2A-2F represent the results of the expression and binding assays of mono-specific scFv to AXL or PD-1 and BsAbl-AXL/PD-1. FIGS. 2A and 2B show Western blot (WB) analysis of scFv-Fc and BsAbl-AXL/PD-1 secretion, respectively, from HEK293F at different time points following transfection. FIGS. 2C-2D and 2E-2F show ELISA detection of the binding of the scFv-Fc and the BsAbl-AXL/PD-1 constructs, respectively, to AXL or PD-1. The secreted scFvs and BsAbl-AXL/PD-1 were diluted at different ratios prior to testing the binding to AXL or PD-1 immobilized on the ELISA plate.

FIGS. 3A-3D represent the results of the expression and binding assays of Bi-1 and Bi-3 bi-specific scFv constructs and monomeric AXL(2) and PD-1(2) scFv-Fc conjugates. FIG. 3A shows WB analysis of mono-specific Abs and BsAbl-AXL/PD-1 constructs secreted from HEK293F 96 hr following transfection. FIGS. 3B and 3C show ELISA detection of the binding of the BsAbl-AXL/PD-1 formats to AXL or PD-1, respectively, compared to the mono-specific Abs. FIG. 3D depicts the results of a surface plasmon resonance (SPR) analysis of the dissociation constant (K_{D}) of each of mono-AXL, mono-PD-1, and one of the bi-specific antibodies (Bi-3), to AXL and PD-1, respectively. FIGS. 4A-4B depict the capacity of the mono-specific and the BsAbl-AXL/PD-1 constructs to PD-1 and AXL expressed on cells by flow cytometry. FIG. 4A demonstrates flow cytometry analysis of Bw cells manipulated to overexpress human PD-1 receptor. The X-axis represents the intrinsic mCherry red fluorescent protein expression in the different Bw cells. Y-axis indicates the expression of PD-1 as demonstrated by reporter green fluorescent protein (GFP) levels. FIG. 4B demonstrates flow cytometry analysis of mono-PD-1 and the bi-specific constructs binding to PD-1 overexpressing cells.

FIGS. 5A-5C depict the properties of BsAb-AXL/PD1 binding to PD-1. FIG. 5A demonstrates ELISA assay results of activation of PD-1 by the commercial antibodies Keytruda or Nivolumab, examined by IL-2 reporter assay. FIG. 5B demonstrates binding results of mono-PD-1 antibody (Ab) and bi-specific constructs to PD-1 expressed on Bw cells. FIG. 5C demonstrates results of blocking the interaction between PD-L1 expressed on A549 cells and PD-1 expressed on Bw cells by mono-PD-1 Ab and bi-specific constructs.

FIGS. 6A-6B represent the binding assay results of two mono-AXL scFv, Enapotamab (AXL1) and YW327.6S2 (AXL2), to AXL knockdown HNSCC cell lines. FIG. 6A demonstrates the binding of monomeric AXL1 and AXL2 to two HNSCC cell lines, SNU1076 and SCC47, with knockdown AXL. FIG. 6B demonstrates flow cytometry analysis of mono-AXL binding to high/low AXL cells.

FIGS. 7A-7C depict in-vitro killing assay of tumor cells by PBMCs in real-time. FIG. 7A represents a series of images taken by the live imager system JuLI Stage, showing the in-vitro killing assay of SCC47 GFP cells (marked as T for target cells) with PBMCs isolated from a healthy donor (marked as E for effector cells) in the presence of mono-specific Abs tested separately (mono-AXL and mono PD-1) and in combination (Combo) compared to the bi-specific formats (Bi-1 and Bi-3). The experiment was conducted in a fixed concentration of the Abs (0.5 nM) and T:E ratio (1:10). FIG. 7B demonstrates cell number analysis by the JuLI STAT software. FIG. 7C demonstrates an ELISA analysis of IFN-7 concentration secreted into the respective cell media (in cell sups) of the cells treated with the different antibodies.

FIG. 8 depicts a flow cytometry analysis of a mix of AXL expressing SCC47 GFP cells and PD-1 overexpressing Bw mCherry cells, incubated with each of no antibodies (negative control), combination of mono-AXL and mono PD-1 antibodies, and Bi-3 antibody, respectively (from left to right). The X-axis represents the Scc47 cells marked by GFP, and the Y-axis indicates the Bw cells marked by mCherry, wherein a double positive represents T-cell-tumor cell doublets.

FIG. 9 depicts the results, measured in tumor growth over time, of in vivo treatment of a mouse model injected with SCC47 cancer cells, and subsequently with PMBCs, and treated with no drugs, combination of mono-AXL and mono PD-1 antibodies, and Bi-3 antibody, respectively.

FIGS. 10A-10B depict the bridging properties of BsAb-AXL/PD1 between AXL and PD-1 expressing cells. FIG. 10A demonstrates a suggested mechanism by which BsAbl-AXL/PD-1 increases the interaction between the PD-1 expressing cell and the AXL-expressing cells, and the effect of monospecific antibodies on the mechanism. FIG. 10B demonstrates the ELISA results for PD-1 activation (indicated by IL2 quantification in supernatant after 24 hr of incubation) in the presence of BsAbl-AXL/PD-1, Combo of mono-AXL and mono-PD-1 Abs, and BsAbl-AXL/PD-1 in the presence of each of the monospecific Abs, respectively.

FIG. 11 depicts a flow cytometry analysis of a mix of AXL expressing FaDu plx GFP cells and PD-1 overexpressing Bw mCherry cells, incubated with each of no antibodies (negative control), combination of mono-AXL and mono PD-1 antibodies, and Bi-3 antibody, respectively (from left to right). The X-axis represents the Scc47 cells marked by GFP, and the Y-axis indicates the Bw cells marked by mCherry, wherein a double positive represents T-cell-tumor cell doublets.

FIGS. 12A-12C depict the bridging effect of BsAb-AXL/PD1. FIGS. 12A-12B demonstrate the bridging experiment analysis in the image stream mix of Fadu plx GFP and Bw overexpressing PD-1 cells stained with DiD. The cells were incubated with BsAb-AXL/PD1, a combination of mono-AXL and mono PD-1 antibodies, and BsAb-AXL/PD1 with each of the mono-antibodies (no drug as negative control. FIG. 12C demonstrates a SEM image of the immune-cancer synapse mediated via BsAb-AXL/PD1 antibody.

FIGS. 13A-13B depict the localizing of the BsAb-AXL/PD1 to tumor region in vivo. FIG. 13A demonstrates time-course (over 96 hr) fluorescence imaging of NSG mice bearing cells derived xenograft (CDX) tumors, comparing control (PBS) and BsAb-AXL/PD1 (5 mg/kg) treated groups (IP). Grayscal stained patches indicate BsAb-AXL/PD1 distribution, with color intensity corresponding to fluorescence signal strength. FIG. 13B demonstrates a fluorescence quantification across an identical region of interest (ROI) for each subject at 24 hour intervals.

FIG. 14 depicts the results, measured in tumor growth over time, of in vivo treatment of a mouse model injected with 4NQO-T, and subsequently treated with no drugs, and murine BsAb-AXL/PD1 antibody at concentrations of 0.1 mg/kg, 1 mg/kg, and 5 mg/kg, respectively.

FIGS. 15A-15D depict the results mice infected with different cancer cells and treated with murine BsAb-AXL/PD1. FIG. 15A demonstrates results of tumor growth over time, of in vivo treatment of a mouse model injected with mEERL cancer cells, and subsequently treated with no drugs, a combination of mono-specific AXL and PD-1 Abs (5 mg/kg), and murine BsAb-AXL/PD1 antibody (5 mg/kg), respectively. FIG. 15B demonstrates a comparison of the endpoint tumor weight results of the treated mouse groups of FIG. 15A. FIG. 15C demonstrates results of tumor growth over time, of in vivo treatment of a mouse model injected with 4NQO-T cancer cells, and subsequently treated with no drugs, a combination of mono-specific AXL and PD-1 Abs (5 mg/kg), and murine BsAb-AXL/PD1 antibody (5 mg/kg), respectively. FIG. 15D demonstrates a comparison of the endpoint tumor weight results of the treated mouse groups of FIG. 15C.

### DETAILED DESCRIPTION OF THE INVENTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. According to some embodiments, the individual is a mammal. According to some embodiments, the mammal is a mouse, rat, rabbit, dog, cat, horse, cow, sheep, pig, goat, llama, alpaca, or yak. According to some embodiments, the individual is a human.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

The terms “cancer” and “tumor” relate to the physiological condition in mammals characterized by deregulated cell growth. Cancer is a class of diseases in which a group of cells displays uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

As used herein the term an “effective amount” refers to the amount of a therapeutic that causes a biological effect when administered to a mammal. Biological effects include, but are not limited to, inhibition or blockade of a receptor-ligand interaction, inhibition of a signaling pathway, reduced tumor growth, reduced tumor metastasis, or prolonged survival of a mammal bearing a tumor. A “therapeutic amount” is the concertation of a drug calculated to exert a therapeutic effect. A therapeutic amount encompasses the range of dosages capable of inducing a therapeutic response in a population of individuals. The mammal can be a human individual. The human individual can be afflicted with or suspected of being afflicted with a tumor.

The term “antibody” herein is used in the broadest sense and includes polyclonal and monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, fully human antibodies, humanized antibodies, and heteroconjugate antibodies, multispecific, e.g., bi-specific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

Bi-specific or Bispecific antibodies (BsAbs) are immunoglobulin constructs with binding sites directed at two different antigens or two different epitopes on the same antigen. According to some embodiments of the present invention, bispecific constructs comprise at least one binding site to human PD1 and at least one binding site to human AXL.

In some embodiments, the bi-specific constructs of the present invention comprise two scFv molecules targeted to the human receptor AXL and two scFv molecules targeted to human PD1. In some embodiments, the bi-specific constructs comprise two polypeptides, each polypeptide comprising two different scFvs and a carrier polypeptide that form a structure selected from scFv(PD-1)-carrier-scFv(AXL) and scFv(AXL)-carrier-scFv(PD-1). In specific embodiments, the bi-specific construct comprises two polypeptides, each polypeptide comprising two different scFvs and a carrier polypeptide, wherein the bi-specific construct forms a structure selected from [scFv(PD-1)]2-IgG1Fc-[scFv(AXL)]2 and [scFv(AXL)]2-IgG1Fc-[scFv(PD-1)]2.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg_N and Rees A P, “WAM: an improved algorithm for modeling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs and bi-specific scFvs.

By the term “single-chain variable fragment (scFv)” is meant a fusion of the V_{H} and V_{L} regions, linked together with a short (usually serine, glycine) linker. Single-chain antibodies can be single chain composite polypeptides having antigen binding capabilities and comprising amino acid sequences homologous or analogous to V_{H} and V_{L} V_{L} (linked V_{H}-V_{L} or a single chain Fv (scFv)). Both V_{H} and V_{L} may copy natural mAb sequences or one or both of the chains may comprise a CDR-FR construct of the type described in U.S. Pat. No. 5,091,513. The separate polypeptides analogous to the V_{H} and V_{L} regions are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. According to some embodiments, the antibody fragments are scFvs.

The term “antigen” as used herein refers to a molecule or a portion of a molecule capable of eliciting antibody formation and being specifically bound by a binding molecule such as an antibody or a fragment thereof comprising the antigen-binding site. An antigen may have one or more than one epitope. The specific binding referred to above is meant to indicate that the antigen will react, in a highly selective manner, with its corresponding antibody and not with the multitude of other antibodies which may be evoked by other antigens. Antigens according to some embodiments of the bi-specific antibodies of the present invention are human AXL and human PD1.

The terms “PD-1” and “PD1” are used interchangeably herein. Unless specified otherwise, the terms include any variants, isoforms and species homologs of human PD-1 that are naturally expressed by cells, or that are expressed by cells transfected with a PD-1 gene. PD-1 proteins include full-length PD-1 (e.g., human PD-1; GI: 167857792; extracellular domain: Pro21-Gln167), as well as alternative splice variants of PD-1, such as PD-1Δex2, PD-1Δex3, PD-1Δex2,3, and PD-1Δex2,3,4. (Nielsen et al., Cellular Immunology, 2005, 235:109-116).

The term AXL refers to a specific protein, which is a member of the Tyro3-Axl-Mer (TAM) receptor tyrosine kinase subfamily (O'Bryan J. P., et al., Mol. Cell. Biol. 1991:11:5016-5031). Unless specified otherwise, the terms include any variants, isoforms and species homologs of human AXL that are naturally expressed by cells, or that are expressed by cells transfected with an AXL gene. The human AXL gene is identified for example as UniProtKB—P30530.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. According to some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences can be determined, including algorithms needed to achieve maximal alignment over the full length of the sequences being compared.

The terms “homologous”, “homology” or “percent homology” when used herein to describe an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, for example.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved antibody-dependent cellular cytotoxicity (ADCC) or complement-dependent cytotoxicity (CDC).

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3, in particular, are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

“Specific binding” or “specifically binds” or “binds” refer to an antibody binding to a specific antigen with greater affinity than for other antigens. Typically, the antibody “specifically binds” when the equilibrium dissociation constant (K_{D}) for binding is about 1×10⁻⁷ M or less, for example about 1×10⁻⁸ M or less, about 1×10⁻⁹ M or less, about 1×10⁻¹⁰ M or less, or about 1×10⁻¹² M or less, typically with the KD that is at least one hundred-fold less than its KD for binding to a non-specific antigen (e.g, BSA, casein). The K_{D} may be measured using standard procedures.

In some embodiments, a scFv provided herein as part of a bi-specific construct has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or less (e.g., 10⁻⁷ M or less, e.g., from 10⁻⁷ M to 10⁻¹² M, e.g., from 10⁻⁸ M to 10⁻¹³ M) for its target, human AXL or human PD1. K_{D} can be measured by any suitable assay. In certain embodiments, KD can be measured using surface plasmon resonance (SPR) assays (e.g., using a BIACORE®-T200, a BIACORE®-4000, a ProteOn XPR36, and the like).

In some embodiments, a bi-specific construct provided herein may be further modified to contain additional non-proteinaceous moieties that are known and available. The moieties suitable for derivatization of the antibody include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the construct may vary, and if two or more polymers are attached, they can be the same or different molecules.

The antibody fragments, conjugates and constructs described herein can be encoded by a nucleic acid. A nucleic acid is a type of polynucleotide comprising two or more nucleotide bases. In certain embodiments, the nucleic acid is a component of a vector that can be used to transfer the polypeptide-encoding polynucleotide into a cell. As used herein, the term “vector” refers to a nucleic acid molecule capable of transporting another nucleic acid to which it has been linked. One type of vector is a genomically integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors, regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

Any human IgG molecule or a portion thereof may be used according to the present invention as a carrier for the two antigen-binding molecules. According to some embodiments, the human IgG is selected from IgG1, IgG2, IgG3 and IgG4 or portions thereof. According to some embodiments, the carrier molecule is a human IgG1 constant region or a potion thereof.

In some embodiments, one or more amino acid modifications may be introduced into the Fc region of a construct provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule. “Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single-stranded or double-stranded. “Expression vector” refers to a vector that can be utilized in a biological system or in a reconstituted biological system to direct the translation of a polypeptide encoded by a polynucleotide sequence present in the expression vector.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not human is a heterologous nucleic acid sequence in that particular context. Whereas heterologous nucleic acids may be derived from a different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g, an exogenous nucleic acid that is foreign to the eukaryotic host cell, or a recombinant nucleic acid that is not normally found in the eukaryotic host cell.

### Therapeutic Methods

In certain embodiments, disclosed herein, are bi-specific constructs useful for the treatment of a cancer or tumor. Treatment refers to a method that seeks to improve or ameliorate the condition being treated. With respect to cancer, treatment includes, but is not limited to, reduction of tumor volume, reduction in the growth of tumor volume, increase in progression-free survival, improvement of overall wellbeing, or overall life expectancy. In certain embodiments, treatment will affect the remission of a cancer being treated. In certain embodiments, treatment encompasses use as a prophylactic or maintenance dose intended to prevent the reoccurrence or progression of a previously treated cancer or tumor. It is understood by those of skill in the art that not all individuals will respond equally or at all to a treatment that is administered, nevertheless these individuals are considered to be treated.

According to some embodiments, the treatment increases the duration of survival of a subject having cancer. According to some embodiments, the treatment increases the progression-free survival of a subject having cancer. According to some embodiments, the treatment increases the response incidence in a group of subjects. According to yet other embodiments, the treatment increases the duration of response of a subject having cancer. According to some embodiments, the treatment prevents or inhibits the development of metastasis in a patient having cancer. According to some embodiments, the treatment prevents tumor recurrence.

In certain embodiments, the bi-specific constructs described herein are for use in the manufacture of a medicament for treating a cancer.

According to a specific embodiment, the invention provides a method of treating cancer in a subject, comprising administering to the subject an effective amount of a bi-specific construct disclosed herein, as a monotherapy or in a combined treatment regimen. According to some embodiments, pharmaceutical compositions comprising the bi-specific constructs of the present invention are administered to a subject in need thereof, in conjugation with additional treatment. According to some embodiments, the additional treatment is a surgery, a radiotherapy, an immunotherapy a chemotherapy, or a combination thereof.

In certain embodiments, the bi-specific constructs described herein are for treating a cancer or tumor that is refractory to a chemotherapy, radiotherapy or immunotherapy treatment. According to some embodiments, the cancer or tumor is refractory to a treatment with a checkpoint inhibitor as a monotherapy. Refractory cancer refers to a cancer/tumor that develops progressive disease despite treatment with the checkpoint inhibitor alone. As used herein “checkpoint inhibitor” refers a drug that inhibits a biological molecule (“checkpoint molecule”) produced by an organism that negatively regulates the anti-tumor/cancer activity of T cells in the organism.

In certain embodiments, the bi-specific constructs comprise a binding molecule, a fragment thereof or the combination of CDR sequences of an antibody that specifically binds PD1, wherein the antibody is selected from Pembrolizumab, Nivolumab, AMP-514, Spartalizumab, and Tislelizumab (BGB-A317).

In certain embodiments, the bi-specific constructs are for use in treating a cancer or tumor. Any cancer or tumor that expresses AXL and/or PD1 is eligible for treatment with the constructs of the present invention. In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, and/or liver tumors. In certain embodiments, tumors which can be treated with the constructs of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more constructs of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer. In certain embodiments, the cancer treated with the constructs of this disclosure is selected from head and neck cell carcinoma (HNSCC) and esophageal squamous cell carcinoma (ESCC). In a certain embodiment, the cancer is refractory to other treatment. According to some embodiments, the cancer is a metastatic cancer.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered, its persistence in the blood circulation, and the judgment of the treating physician.

In certain embodiments, the bi-specific constructs can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the bi-specific constructs are administered intravenously. In certain embodiments, the bi-specific constructs are administered subcutaneously. In certain embodiments, the antibodies are administered intratumorally. In certain embodiments, the bi-specific constructs are administered on a suitable dosage schedule, for example, weekly, twice weekly, monthly, twice monthly, once every two weeks, once every three weeks, or once a month etc. In certain embodiments, the bi-specific constructs are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 2 mg/kg and about 30 mg/kg. Therapeutically effective amounts include amounts are those sufficient to ameliorate one or more symptoms associated with the disease or affliction to be treated.

The pharmaceutical composition according to the present invention may be administered together with an anti-neoplastic composition. According to a specific embodiment, the anti-neoplastic composition comprises at least one chemotherapeutic agent. The chemotherapeutic agent, which could be administered separately or together with the antibody according to the present invention, may comprise any such agent known in the art exhibiting anti-cancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate. According to a specific embodiment, the chemotherapeutic agent is selected from the group consisting of alkylating agents, antimetabolites, folic acid analogs, pyrimidine analogs, purine analogs and related inhibitors, vinca alkaloids, epipodopyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the antibody or fragment thereof.

### Pharmaceutically Acceptable Excipients, Carriers, and Diluents

In certain embodiments, the bi-specific constructs of the current disclosure are included in a pharmaceutical composition comprising one or more pharmaceutically acceptable excipients, carriers, and diluents. The excipients or other additives must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired exposure.

In certain embodiments, the bi-specific constructs of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

Typically, the bi-specific constructs of the present invention will be suspended in a sterile saline solution for therapeutic uses. The pharmaceutical compositions may alternatively be formulated to control release of active ingredient (molecule comprising the antigen-binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of an antibody or antibody fragment, from such a matrix depends upon the molecular weight of the molecule, the amount of the molecule within the matrix, and the size of dispersed particles.

In certain embodiments, the bi-specific constructs of the current disclosure are shipped/stored lyophilized and reconstituted before administration. In certain embodiments, lyophilized formulations comprise a bulking agent such as mannitol, sorbitol, sucrose, trehalose, dextran 40, or combinations thereof. The lyophilized formulation can be contained in a vial comprised of glass or other suitable non-reactive material. The compositions when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

Also described herein are kits comprising one or more of the bi-specific constructs described herein in a suitable container and one or more additional components selected from: instructions for use; a diluent, an excipient, a carrier, and a device for administration.

In certain embodiments, described herein is a method of preparing a cancer treatment comprising admixing one or more pharmaceutically acceptable excipients, carriers, or diluents and a bi-specific construct of the current disclosure. In certain embodiments, described herein is a method of preparing a cancer treatment for storage or shipping comprising lyophilizing one or more constructs of the current disclosure.

### EXAMPLES

### Materials and Methods

### ELISA:

For binding PD-1: ELISA 96-well plates were coated with Streptavidin (NEB) followed by incubation with biotinylated human PD-1 recombinant protein (ACROBiosystems). The plates were then blocked, and incubated with serial dilutions of the antibodies. Next, anti-human-FC-HRP (Jackson laboratories) was added to the plates and a reaction was developed with TMB substrates. Once color developed H2SO4 was used to end the reaction. Plates were washed with PBST (PBS containing 0.05% Tween-20) between the steps. Each of the mentioned steps was carried out for 1 hour at room temperature. The plates were then read on a spectrophotometer at 450 nm.

For binding AXL: MaxiSorb 96-well plates were coated with human Axl recombinant protein (ACROBiosystems). The subsequent steps were performed as in the ELISA for binding PD-1 (blocking, antibodies, anti-human-FC-HRP and TMB).

For binding IFN-γ: The assay was performed using human IFN-γ Standard TMB ELISA Development Kit from PeproTech (Catalog Number: 900-T27) according to the manufactures' protocol. In brief, 96 ELISA plates were coated with capture antibody overnight. Next day, a blocking step was performed and then culture supernatants, as well as protein standard, were applied to the plate. Then, the plate was incubated with detection antibody followed by another half an hour with HRP-Conjugate antibody. Lastly, TMB substrate was added for color development and stop solution was applied. Quantification was carried out by protein standard curve calculated by reading at 450 and 620 nm wavelengths in a spectrophotometer.

### Example 1: Design, Cloning, Expression, and Binding Analysis of Mono-Specific scFv Against AXL and BsAbl-AXL/PD-1

### Design, Production, and Initial Tests of Anti-AXL-scFv

several scFv clones that bind AXL are identified produced using scFv library in yeasts. After validation that the anti-AXL-scFv is expressed in yeast and binds to its target, the yeast anti-AXL-scFvs library is created. The library of 10⁷-10⁹ anti-AXL-scFvs variants, is generated by a random mutagenesis process using error-prone PCR. Following PCR, a transformation of the library using pETCON2 plasmid into EBY100 competent yeast is performed based on homologous recombination. A library containing between 2-5 random mutations per gene is produced to allow the identification of beneficial mutations in the anti-AXL-scFv.

The yeast library containing the anti-AXL-scFvs variants is then screened for the desired properties. Specifically, FACS is used for sorting the variants that present high expression levels and binding to the target, meaning a high anti-c-Myc and anti-His signal. Three cycles of FACS sorting with different ligand concentrations of the AXL recombinant protein are performed, starting with a relatively high concentration of the ligand to sort for cells showing the best expression and binding signal. These variants are grown and subjected to consecutive rounds of FACS sorting with a lower AXL ligand concentration. Following three cycles of sorting, individual clones of variants that exhibit improved expression and binding properties relative to the WT anti-AXL-scFv are identified.

Antibody sequence and bioinformatics tools were used to predict the scFv sequences for the generation of molecules targeting AXL and PD-1 (mono-specific constructs). Specifically, Enapotamab (AXL1) and YW327.6S2 (AXL2) sequences that target AXL and Keytruda (PD-1-1) and Nivolumab (PD-1-2) that target PD-1 were utilized. The chosen sequences were cloned into the pFUSE-Fc-His plasmid using Gibson assembly. The pFUSE-hIgGle3-Fc2 plasmid (InvivoGen) is a human IgG1 engineered Fc that comprises the CH2 and CH3 domains of the IgG heavy chain and the hinge region.

For AXL, following successful cloning, HEK293F cells were transfected with the pFUSE-anti-AXL-scFv-Fc gene and FreeStyle™ 293 expression media was used to produce a small amount of the anti-AXL-scFv. Using this approach, the scFv is secreted to the FreeStyle™ 293 media due to an IL-2 signal peptide fused to the N-terminus of anti-AXL-scFv. In this way, the protein can be easily collected and purified for further analysis. Next, an initial characterization of the anti-AXL-scFv was performed. The secretion into the media was tested using WB followed by ELISA to test for AXL binding. In addition, the binding of anti-AXL-scFv to native AXL on tumor cells surface was tested. Several tumor cell lines with a diverse expression of AXL have been developed and used (Badarni et al. ibid). Specifically, the SCC47 cell line that expresses AXL and the variant with knockdown of AXL, shAXL-cells, were used for flow cytometry analysis.

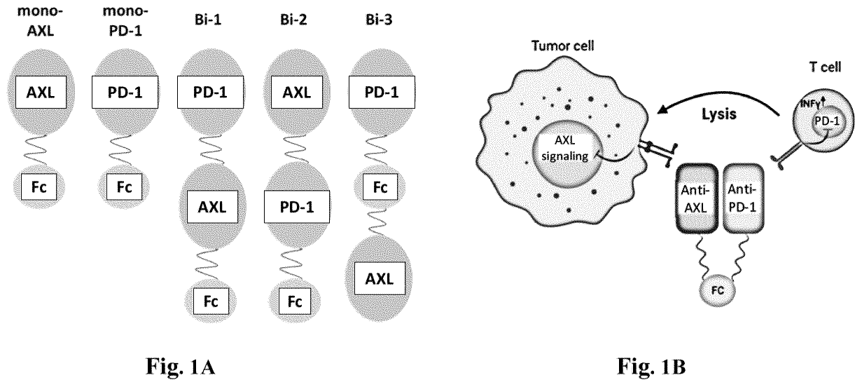

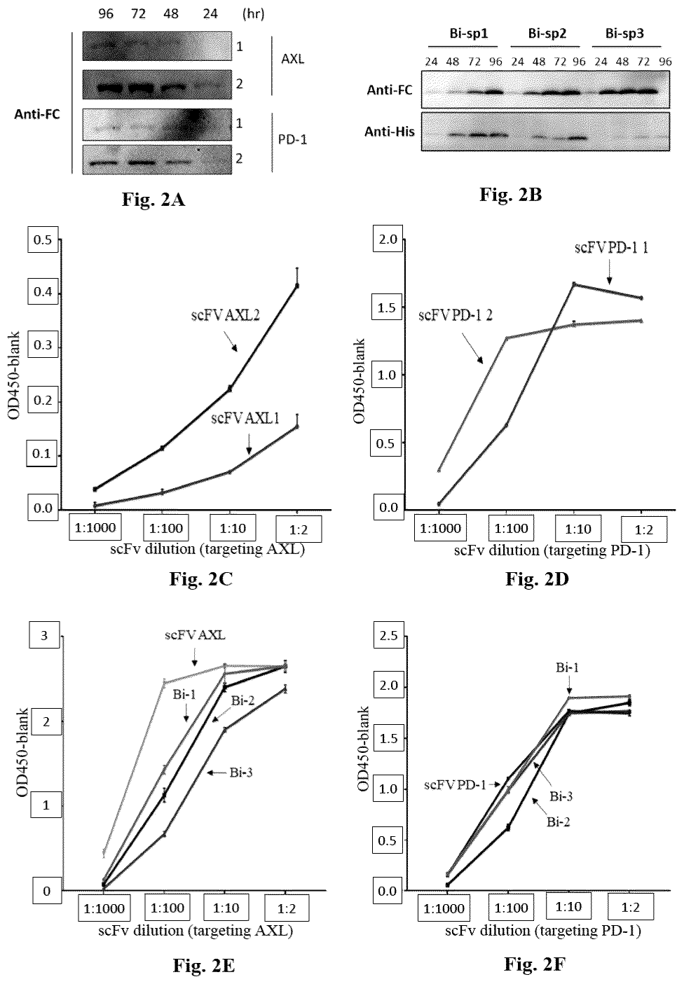

Based on the mono-specific inhibitors, and by using the same technique, bi-specific scFv inhibitors (BsAbI) targeting AXL and PD-1 simultaneously, namely as BsAbl-AXL/PD-1 were designed and cloned. Since it is impossible to predict how the spatial configuration of BsAbl-AXL/PD-1 will affect binding and efficacy, three configurations were generated as shown in FIG. 1A. Similar to the generation of the mono-specific scFvs, two chosen scFv were cloned in tandem (Bi-1 and Bi-2) and at two sides of the Fc (Bi-3) into the pFUSE-Fc-His plasmid (pFUSE-hIgGle3-Fc2, InvivoGen), forming the BsAbl-AXL/PD-1 (FIG. 1A). To produce the two mono-specific Abs and three bi-specific Abs, HEK293F cells were transfected with different pFUSE vectors corresponding to the mono or bi-specific inhibitor sequences and the FreeStyle™ HEK293 media was used to produce a small amount of each mono-specific scFv-Fc and BsAbl-AXL/PD-1. The secretion of scFv-Fc and BsAbl-AXL/PD-linto the media was tested using WB against Fc domain (FIG. 2A) or against Fc domain and His tag that is fused to the C terminal of the Abs (FIG. 2B) for up to about 96 h following transfection.

### Binding Tests and Further Antibody Production

ELISA was performed to test the binding capacity of the scFv-Fc to their targets (FIGS. 2C and 2D). The anti-AXL and anti-PD-1 scFv-Fc were tested against human AXL protein and human PD-1 protein, respectively. Based on the WB and ELISA results (FIGS. 2A and 2C-2D, respectively), AXL2 and PD-1-2 scFv-Fc were selected for further development of BsAbl-AXL/PD-1. ELISA binding characterization of the developed BsAbl-AXL/PD-1 variants validated the targeting of all five constructs to AXL and PD-1 (FIG. 2E-2F).

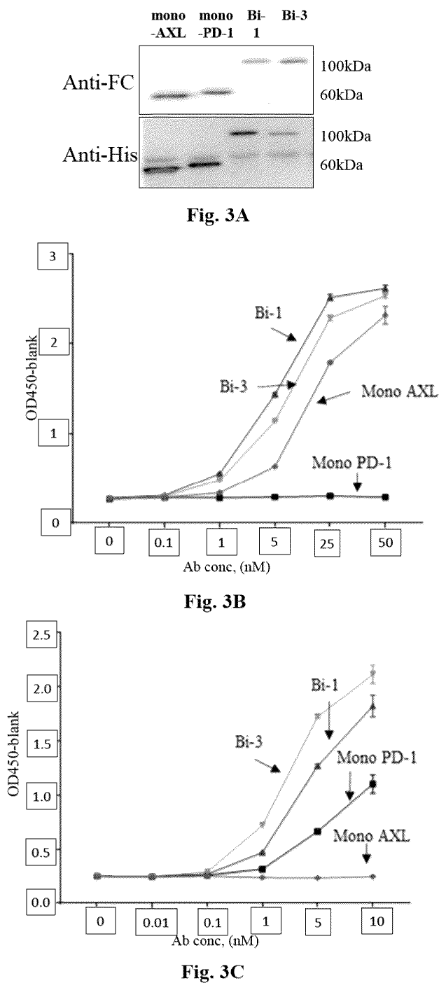

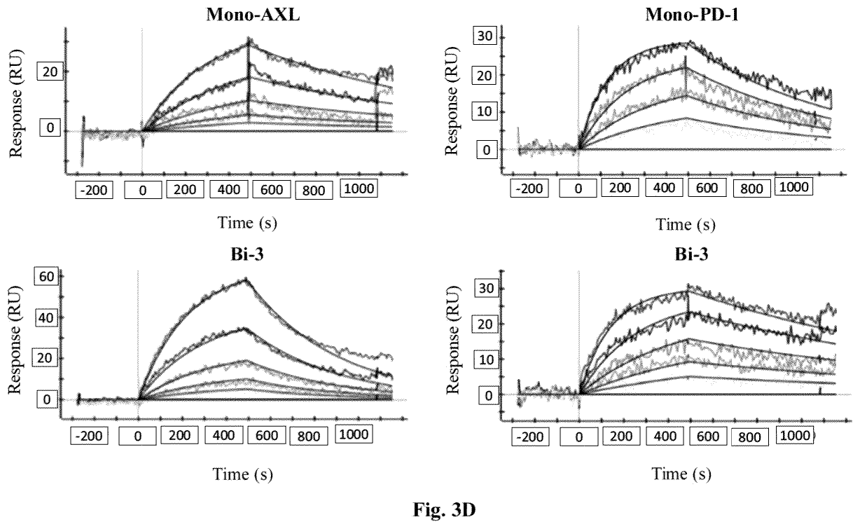

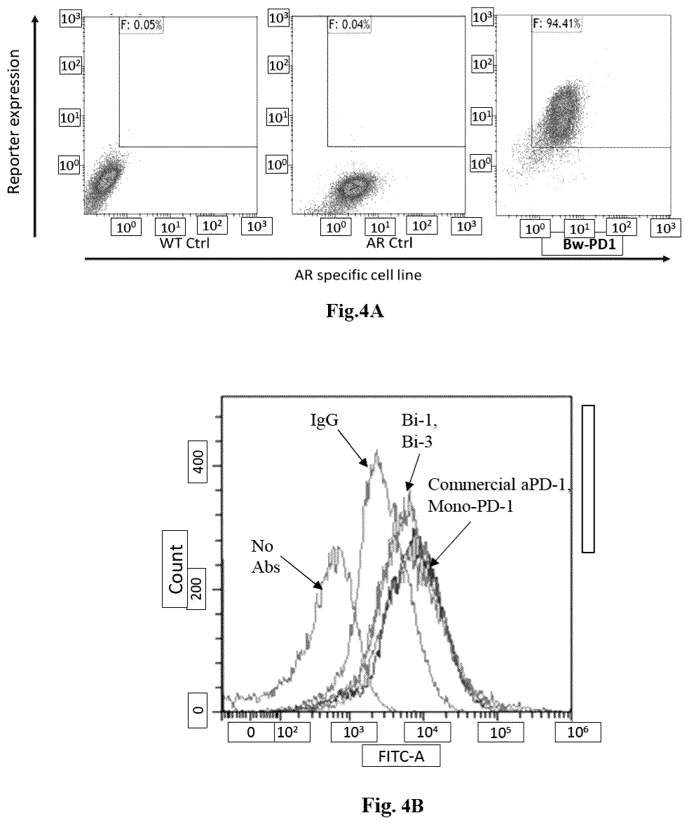

Bi-1 and Bi-3 bi-specific constructs and two mono-specific Abs, AXL2 and PD-1-2 scFv-Fc, were selected for further medium-scale production using the FreeStyle™ HEK293 media according to Weizman et al. (ront Mol Biosci. 2017; 4(AUG):61) and Koslawsky et al. (Oncotarget. 2018; 9(47), 28500). In this expression system, the inhibitors are secreted to the FreeStyle™ media and are easily collected and purified for further analysis. The amount of inhibitor secreted into the media was tested after 96 hr following transfection, using WB against the Fc or His tag (FIG. 3A).

All four constructs were purified using Ni-NTA resins and diluted to different concentrations prior to ELISA binding analysis. Recombinant AXL and PD-1 were used for testing the binding of the BsAbl-AXL/PD-1 to their targets in comparison to the mono-specific controls. As is shown in FIG. 3B-3C, increased inhibitor concentration enhanced the binding to the recombinant protein targets. The ELISA analysis further showed that BsAbl-AXL/PD-1 constructs, both Bi-1 and Bi-3, bind AXL and PD-1 even better than the corresponding mono-specific controls (FIG. 3B-3C). To further determine the dissociation constant (K_{D} value) of the mono specific and bi-specific antibodies to their respective targets, the surface plasmon resonance (SPR) method was utilized (using Biorad ProteOn XPR36). The different antibodies were immobilized in independent channels on a SPR sensor prism (ProteOn XPR36 compatible chip), as ligands. Several solutions containing different concentrations of recombinant AXL and PD-1 proteins, as analytes, were streamed into the system in turn (50 nM, 25 nM, 12.5 nM, 6.25 nM and 3.125 nM (i.e., sequential halving), from top to bottom, respectively as demonstrated in FIG. 3D). Based on the association and dissociation interaction between each of the recombinant proteins and the respective antibodies, respective K_{D} values were calculated. The K_{D} for Bi-3-AXL complex was calculated at 60 nM (ka:4.11E+04 1/Ms; kd:2.47E-03 1/s; KD:6.00E-08 M), while for mono-AXL Abs binding AXL it was calculated at 20 nM (ka:4.90E+04 1/Ms; kd:1.02E-03 1/s; KD:2.09E-08 M). With regard to the PD-1 protein, a 5 nM K_{D} was calculated for both mono-PD-1-PD-1, and Bi-3-PD-1 complexes ((ka:2.49E+05 1/Ms; kd:1.48E-03 1/s; KD:5.92E-09 M) and (ka:1.28E+05 1/Ms; kd:7.43E-04 1/s; KD:5.82E-09 M), respectively).

Taken together, the results shown in FIGS. 3B-3D demonstrate that the binding of the bi-specific constructs Bi-3 and Bi-1 to the recombinant AXL and PD-1 proteins is substantially equal to, and under some conditions even better than, the binding of the respective mono antibodies.

### Example 2: Binding Capacity of the BsAb-AXL/PD1 to T Cells and HNSCC Cells

The binding capacity of the mono-specific and the BsAbl-AXL/PD-1 formats to PD-1 and AXL expressed on Bw cells was tested by flow cytometry. Specifically, for testing the binding to PD-1 receptor, a recently generated artificial reporter (“AR”) T cell line with overexpression of PD-1, was used (“Bw-PD-1”) (FIG. 4A). The Bw-PD-1 cells were found to overexpress PD-1 compared to Bw AR control cells (“AR Ctrl”), and to wildtype cells.

Flow cytometry analysis of mono-PD-1 and the bi-specific formats binding to PD-1 overexpressing cells was conducted (FIG. 4B). The mono and bi-specific Abs were detected with Alexa Fluor® 488 anti-human Fc. For the negative control, only the secondary antibodies, Alexa Fluor® 488 anti-human Fc, were used as IgG control. For another negative control, Bw-PD-lcells were used without antibodies (designated “No Abs”). The mono-PD-1, Bi-1 and Bi-3 were found to bind PD-1 receptors on Bw-PD-1 cells at a similar level, (compared to the negative control baseline) indicating that the bi-specific formats retain their binding capacities to PD-1 even after adding the anti-AXL domain. Moreover, the PD-1 binding signal was similar to a commercial anti-PD-1 Ab (FIG. 4B).

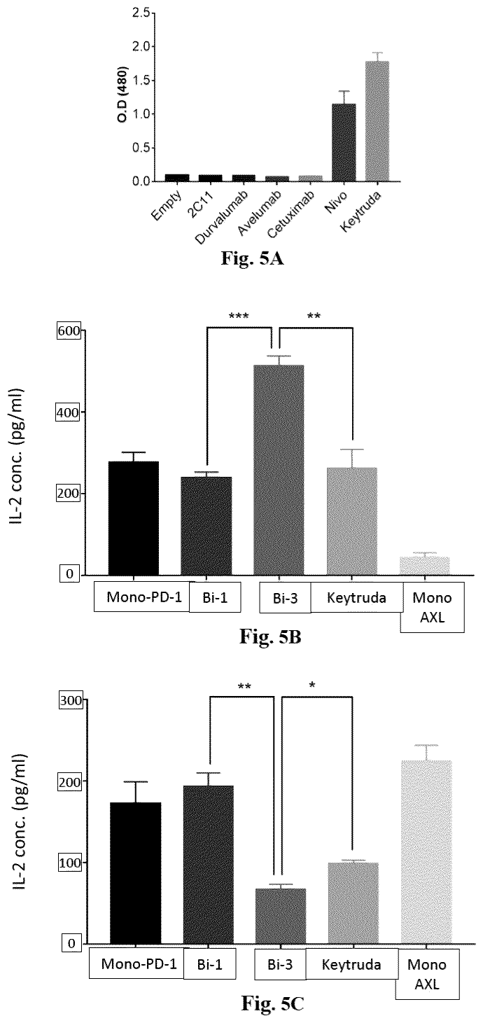

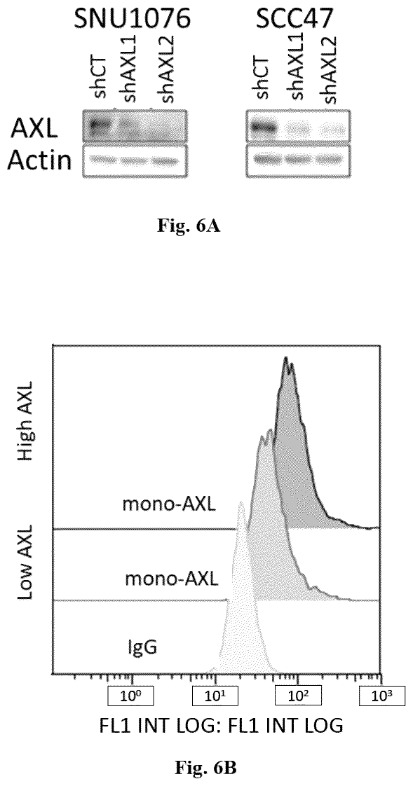

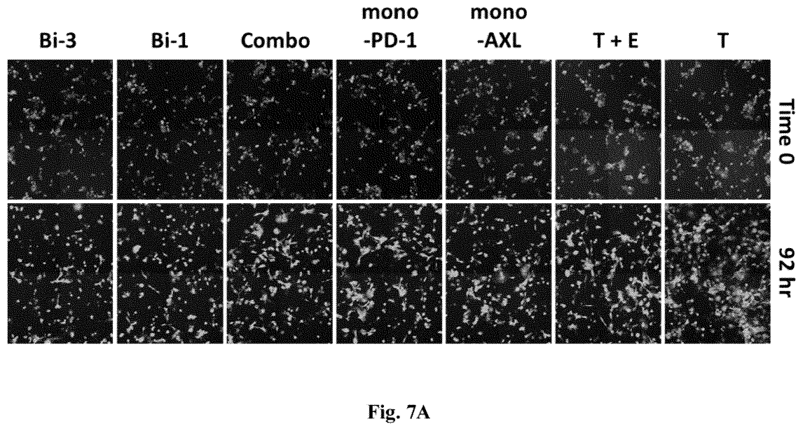

The Bw-PD-1 is a reporter cell line, originated from murine T cell line BW5147 that was manipulated to overexpress human PD-1 receptor, and therefore can be used to measure PD-1 activation, using ELISA, by the levels of secretion of IL-2. The Bw-PD-1 was generated by a fusion of PD-1 with a zeta chain to induce IL-2 as a reporter. As shown in FIG. 5A, only plates coated with Nivolumab and Keytruda (two commercial anti PD-1 antibodies) were found to activate cells to produce IL-2. Using the Bw-PD-1 cell line presented in FIG. 4A, the binding of the mono-specific and bi-specific Abs to the PD-1 reporter cell line was assessed (FIG. 5B) as well as the ability of mono-specific and bi-specific constructs to prevent binding of the PD-1 reporter cell line to the ligand, PD-L1 (FIG. 5C). Keytruda and mono-AXL were used as positive and negative controls, respectively. As can be seen in FIG. 5B, the mono-specific and bi-specific inhibitors, at a 1:10 dilution, show high IL-2 secretion, indicating PD-1 binding, at least at the level of the commercial Ab Keytruda. Surprisingly, the bi-specific Bi-3 Ab displayed an IL-2 secretion at a level significantly higher than that produced by the presence of Keytruda, suggesting that Bi-3 binds and activates PD-1 to secrete IL-2 even better than commercial PD-1 antibodies. Moreover, upon addition of the ligand PD-L1 into the medium, the highest ability to inhibit the interaction of PD-1 with its ligand PD-L1, indicated by a low IL-2 secretion, was found for the construct Bi-3, also compared to Keytruda (FIG. 5C). Regarding the binding to AXL, recently developed HNSCC tumor cell lines with diverse expression levels of AXL, SNU1076 and SCC47 that express AXL (designated “shCT”) (Badarni et al., JCI Insight. 2019; 4(8):e125341), and the variant shAXL-cells with knockdown of AXL, were used (FIG. 6A). Using flow cytometry, the binding of mono-AXL Fc conjugates Enapotamab (AXL1) and YW327.6S2 (AXL2), to SCC47 cells was tested with high and low AXL expression. The mono-AXL Fc conjugates were detected with Alexa Fluor® 488 anti-human FC. The results showed enhanced binding of Mono-AXL conjugate to SCC47 shCT cells (designated “High AXL”), compared to shAXL cells with knockdown of AXL (“Low AXL”) (FIG. 6B).

Next, the Bw-PD-1 cell line was used to further assess the binding of PD-1 receptor by the monospeicifc and bispecific antibodies. FaDu HNSCC cells were seeded, and 24 hours later, Bw-PD-1 cells and the respective antibodies (Bi-3; combination of mono-AXL and mono-PD-1 (“Combo”); Bi-3 and mono-AXL; and Bi-3 and mono-PD-1) were added. A schematic representation of the experiment is shown in FIG. 10A. The antibodies were added at several different concentrations (namely lnM, 5 nM, or 25 nM), as indicated in the figures (see FIG. 10B). After a 24-hour incubation, the media was collected and subjected to an ELISA assay for quantifying IL-2 levels therein. To enhance specificity, the experiment was conducted in the presence of PD-L1 and PD-L2 blockers, ensuring that IL-2 levels would only indicate the interaction of PD-1 receptor with anti-PD-1 antibodies. The results are shown in FIG. 10B.

As can be seen, substantially no signal was quantified in the presence of the two single-arm antibodies (Combo), even at the 25 nM concentration. However, in the case of Bi-3, high IL-2 levels were detected, rising in a concentration-dependent manner. When testing whether the presence of mono-specific antibodies compromises the Bi-3 binding effect, it was found that both mono-AXL and mono-PD-1 acted as binding competitors leading to somewhat reduced IL-2 secretion. Notably, whereas mono-AXL produced a relatively small reduction, mono-PD-1 exhibited strong competitiveness, leading to a substantial reduction in the IL-2 secretion achieved by Bi-3. However, when Bi-3 was present in excess relative to mono-PD-1, the elevated IL-2 secretion levels were maintained. This strengthens the conclusion that mono-AXL and mono-PD-1 can neutralize Bi-3, preventing it from binding both to the cancer cell and to the immune cell.

### Example 3: In-Vitro Killing Assay of Tumor Cells by PBMCs in Real-Time

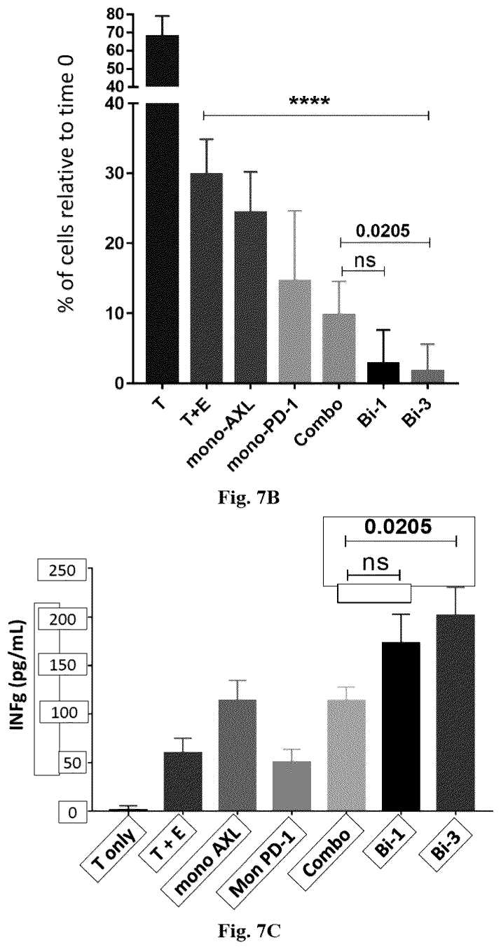

A co-culture system of human HNC cell lines and native PBMCs was established to measure the killing of tumor cells by PBMCs in real-time (FIG. 7A). This imaging system enables measuring the killing kinetics and the interaction between tumor cells and PBMCs. Specifically, SCC47 GFP cells were used as targets and PBMCs isolated from a healthy donor were used as effectors in a ratio of 1:10 (target: effector). The effect of the mono-specific conjugates was tested separately (mono-AXL and mono PD-1) and in combination (Combo) compared to the bi-specific constructs (Bi-1 and Bi-3) in the presence of PBMCs. The experiment was conducted in a fixed concentration of the scFv constructs (0.5 nM). The images were taken by the live imager system, the JuLI Stage. As can be shown in FIG. 7A, there is a clear difference between the Bi-1 and Bi-3 relative to the other samples at 92 hr. The analysis was conducted (n=25 per sample) at time zero compared to the endpoint (92 hr).

FIG. 7B demonstrates cell number analysis by the JuLI STAT software. The results show that the mono and the bi-specific constructs substantially enhanced tumor cell killing. Moreover, the Bi-3 construct was found to be significantly superior to the combination therapy with the two mono-specific Abs.

FIG. 7C demonstrates ELISA measurement of IFN-γ concentration (as explained in the “Materials and Methods” section above), present in the cell culture supernatants of the killing assay of FIG. 7A. The results show that the presence of mono-AXL antibody, combination of the mono-specific Abs, and the bi-specific constructs in the killing assay, respectively, was correlated with elevated IFN-γ concentrations. Moreover, interferon-7 levels were found to be enhanced in the presence of the bi-specific constructs, and particularly the Bi-3 construct, significantly more than in the presence of the combination of the two mono-specific Abs. Activation of T cells usually results in increased IFN-γ secretion, suggesting that the increased killing observed with the Bi-3 construct is probably due to stronger T cell activation, reflected by higher interferon-7 levels.

### Example 4: Bridging Capacity of BsAb-AXL/PD1 (Bi-3) Between T Cells and HNSCC Cells

The effect of the mono-specific and the Bi-3 antibodies, respectively, on the coupling between the cancer cells and the effector cells (herein “bridging capacity”) was tested using a flow cytometry analysis (BECKMAN, Cytoflex). A mix of AXL expressing SCC47 GFP cells and PD-1 overexpressing Bw mCherry cells was incubated with no antibodies (negative control), combination of mono-AXL and mono PD-1 antibodies, and Bi-3 antibody, respectively, for 2 hours at 4° C. Following incubation cells were analyzed by flow cytometry, where the X-axis represents the SCC47 cells marked by GFP, and the Y-axis indicates the Bw cells marked by mCherry. A double positive (GFP and mCherry) represents T-cell-tumor cell (or cancer cell-effector cell) doublets.

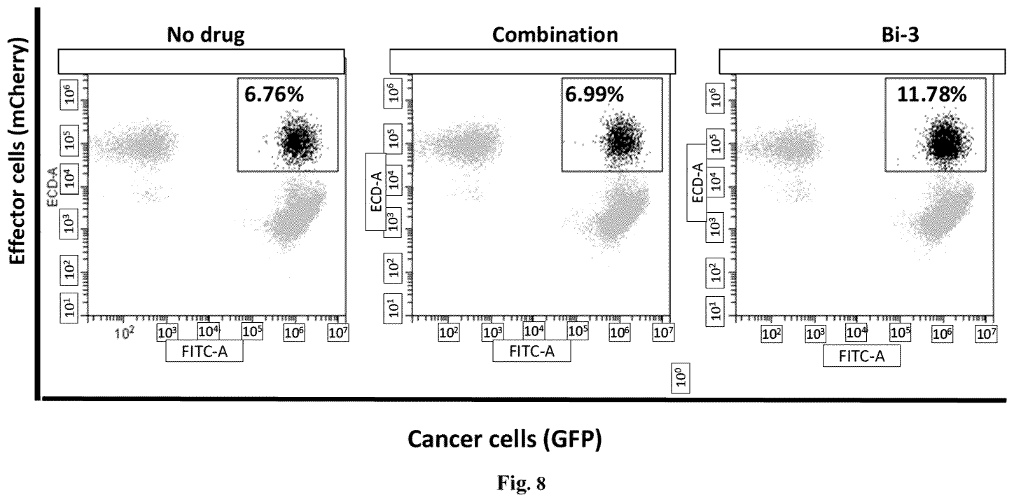

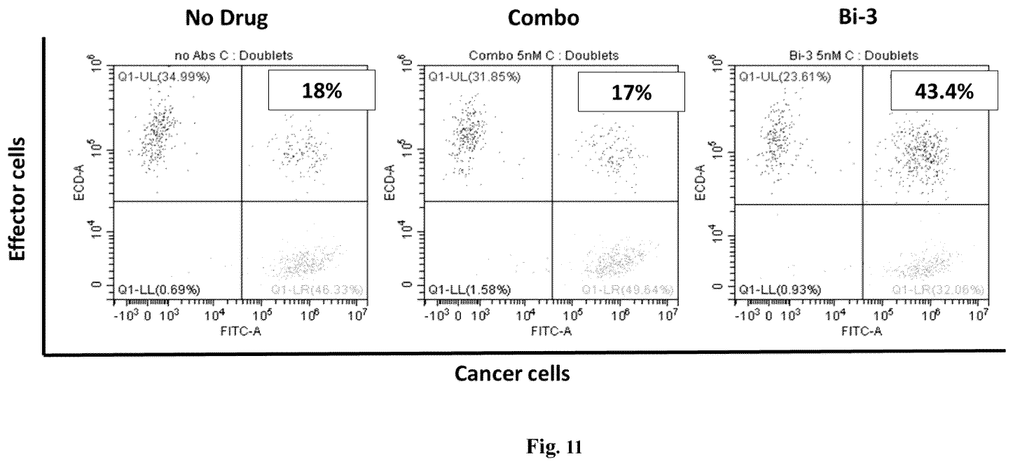

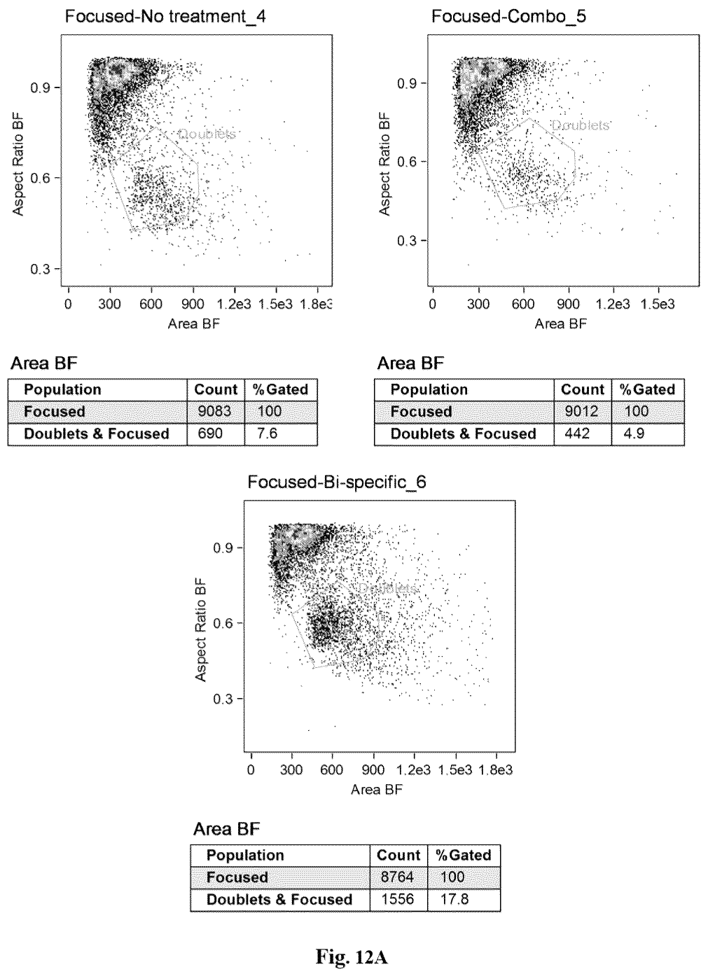

As can be seen in FIG. 8, The incubation with a combination of mono antibodies was found to have a similar coupling rate to incubation with no antibodies. In contradistinction, incubation with BsAb-AXL/PD1 (Bi-3) was found to produce a significantly higher bridging rate between the cancer and the immune cells (74% and 68% increase, as compared to no Abs and combination mono Abs, respectively). The bridging capacity experiment was then repeated, exactly as described with relation to FIG. 8, except that the tested cancer cells were AXL expressing FaDu GFP cancer cells (instead of SCC47 cancer cells). As can be seen in FIG. 11, the incubation with a combination of mono antibodies was again found to have a similar coupling rate to incubation with no antibodies. Incubation with Bi-3, on the other hand, enhanced the interaction between AXL-tumor cells and PD-1-expressing T cells by 2.5-fold as compared to the combination of the two mono-targets antibodies and to the baseline levels without treatment.

Without wishing to be bound by any theory or mechanism of action, this enhanced coupling may assist the effector cells in targeting and subsequently killing the cancer cells, as shown in the results of FIGS. 7A to 7C above.

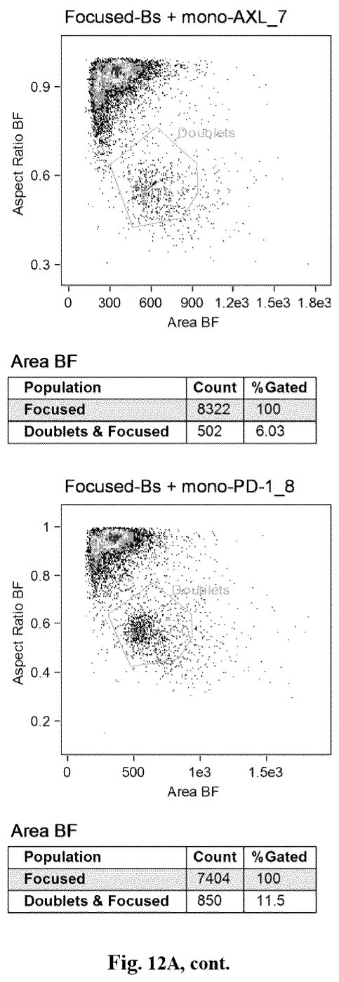

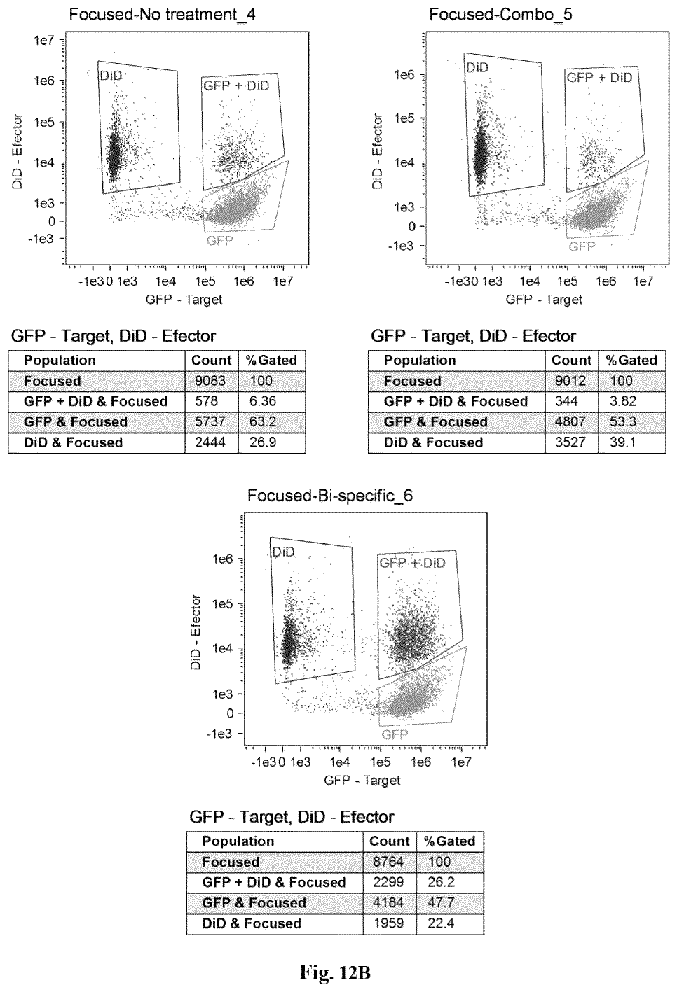

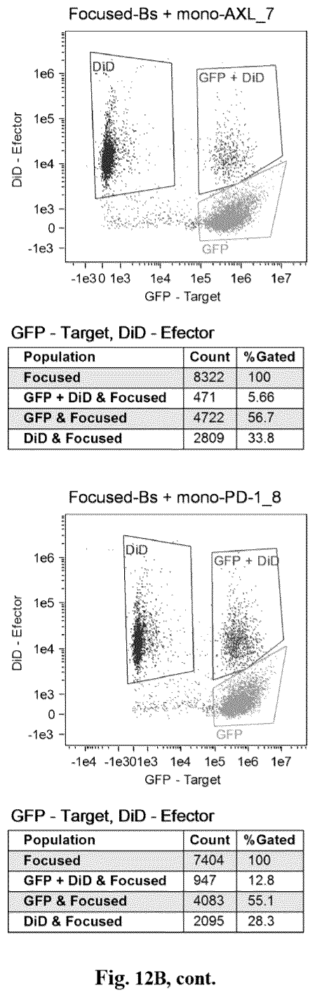

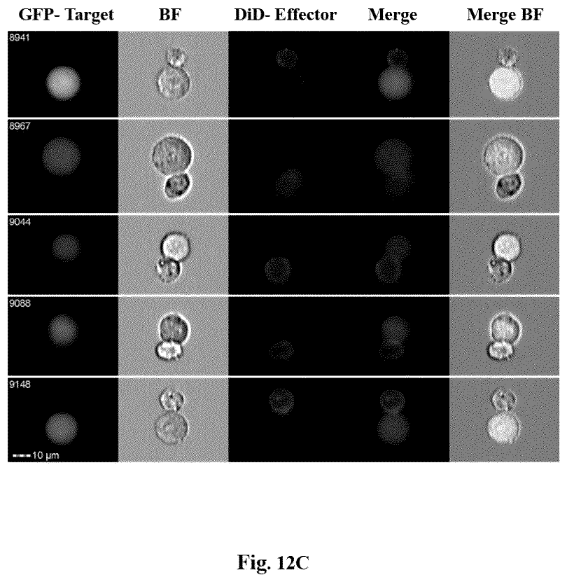

Next, image stream technology was utilized to visualize the immune-cancer cell synapse. A mix of FaDu-plx GFP cancer cells and Bw-PD-1 cells, stained with DiD, were incubated with Bi-3 antibody, a combination of mono-AXL and mono PD-1 antibodies, or with Bi-3 with each of the mono-antibodies, respectively (no drug was used as a negative control). The results of the image-stream are presented in FIGS. 12A-12B. As can be seen, treatment with Bi3 substantially increased the number of immune cell-cancer cell doublets, which increase was somewhat compromised by the addition of each of the mono-specific antibodies. This further implies that the bi-specific antibody effectively promotes a physical interaction between T cells and tumor cells. A visual image of the coupling of the GFP-marked cancer cell and the mCherry-stained Bw-PD-1 cells, in the presence of Bi-3, is shown in FIG. 12C.

### Example 5: In Vivo Treatment of HNSCC Tumor Cells with PMBCs and Added Antibodies

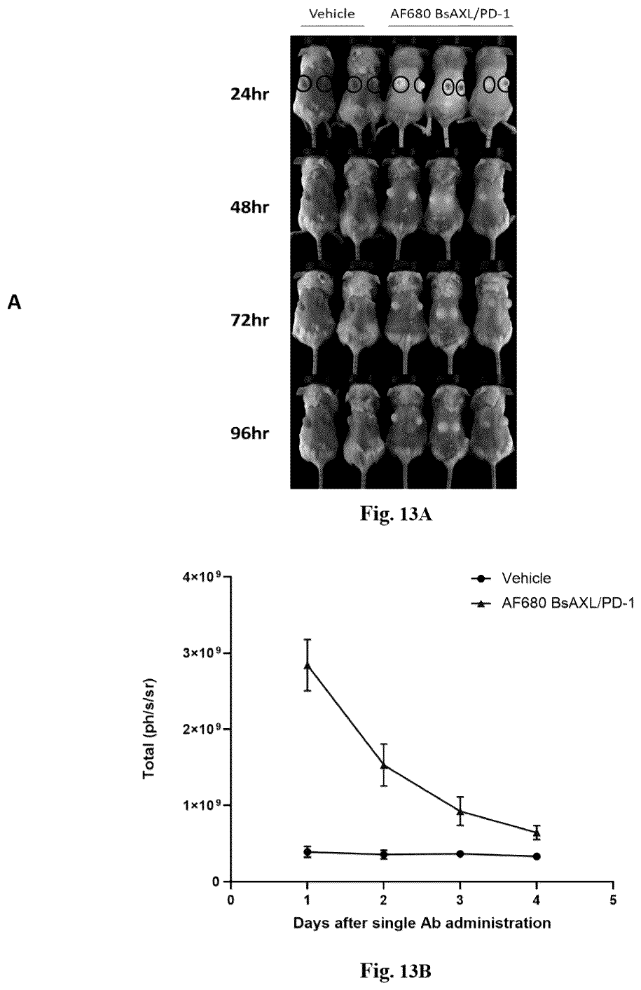

A mouse cancer model was established to test the effect of the bi-specific constructs on the tumor growth of a cell derived xenograft (CDX) in vivo. As a preliminary stage, to assess the targeted delivery capabilities of Bi-3, the antibody was conjugated with Alexa-Fluor 680, and were injected into NSG mice bearing AXL-expressing cell-derived xenografts (CDX) from FaDu cancer cells. The Newton imaging system was utilized to visualize the bio-distribution of Bi-3 in the treated mice for 4 days, at 24 hour intervals. As can be seen in FIGS. 13A-13B, the Bi-3 antibody was detected at the tumor site (indicated in the figure by a white hoop encircling thereof) within 24 hours post-administration, with subsequent reductions in signal intensity over four days, indicating effective tumor targeting and accumulation.

Next, 6-week-old male NSG mice were randomized into 3 groups (n=3) and were injected subcutaneously in the flank with 2×10\hat{ }6 AXL-expressing SCC47 cells in 100 μl PBS (100 μl in each side). After seven days the mice were injected with human PMBCs (isolated form healthy donors) into the tail vein, following which each group was administered a different treatment: a group with no drugs (control group), a group given combination of mono-AXL and mono PD-1 antibodies, and a group given Bi-3 bi-specific antibodies. The treatment was delivered into the mice via intraperitoneal injection twice a week, at a concentration of 2 mg antibodies/kg mouse-weight. The tumor volume was measured over the course of 13 days using a digital caliper, according to the formula V=length×width2×(π/6).

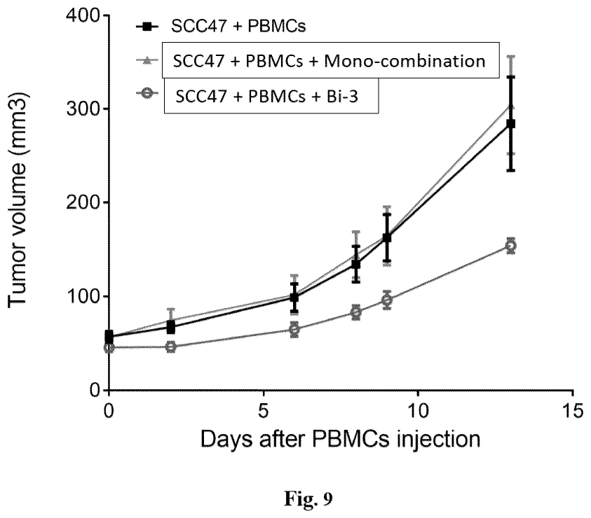

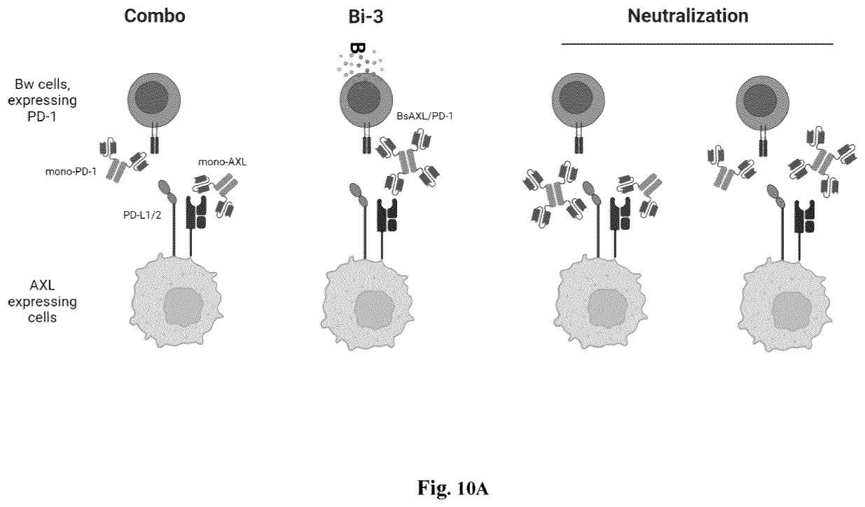

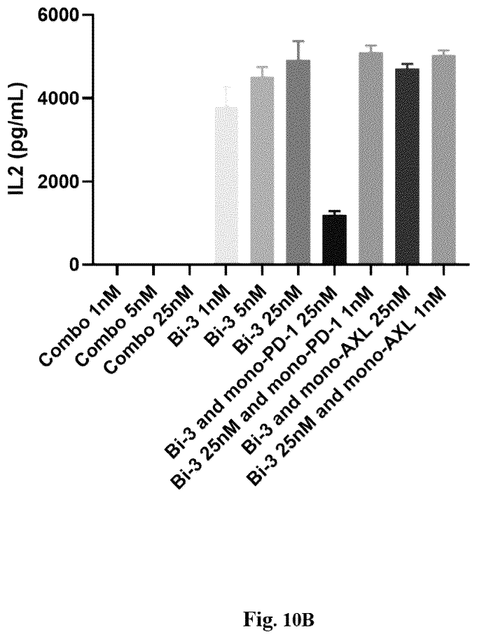

As can be seen in FIG. 9, at the outset the tumor volume was substantially the same in all three groups (about 50-60 mm³). During the course of treatment, the tumor grew to about 300 mm³ in both the control group and the group treated with combination of mono-AXL and mono PD-1 antibodies. In the Bi-3-treated group, on the other hand, the tumor displayed a much slower growth rate, reaching about 130 mm³ at the end of the 13 days. This indicates a substantial inhibitory effect of the Bi-3 antibodies on the progression of HNSCCs in vivo, suggesting a potential therapeutic effect of Bi-3 for cancer patients.

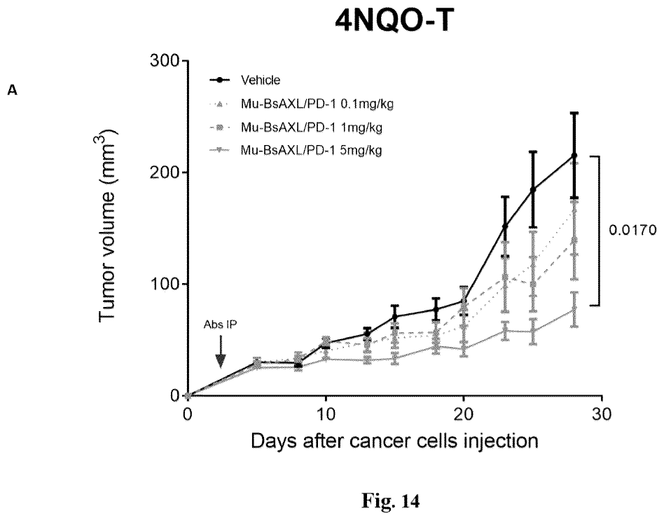

To further assess the anti-cancer effect of the bispecific antibodies, further in vivo studies were performed in immunocompetent WT mice using two additional murine cancer cell lines. First, a dose-response study involving 4NQO-T cancer cells in a sub-cutaneous CDX model was performed. Mouse groups were treated with murine bi-specific antibodies targeting AXL and PD-1 (Mu-BsAb-AXL/PD-1) at concentrations of 0.1, 1, and 5 mg/kg, respectively. The first dosage of antibodies was delivered at two days following cancer-cell injection, and additional dosages were delivered twice-weekly for 28 days. The results representing the progression of tumor growth are presented in FIG. 14. As can be clearly seen, inhibition of tumor growth was achieved across all concentrations at a level which was dose-dependent, with significant inhibition observed at 5 mg/kg compared to control (vehicle-treated) mice.

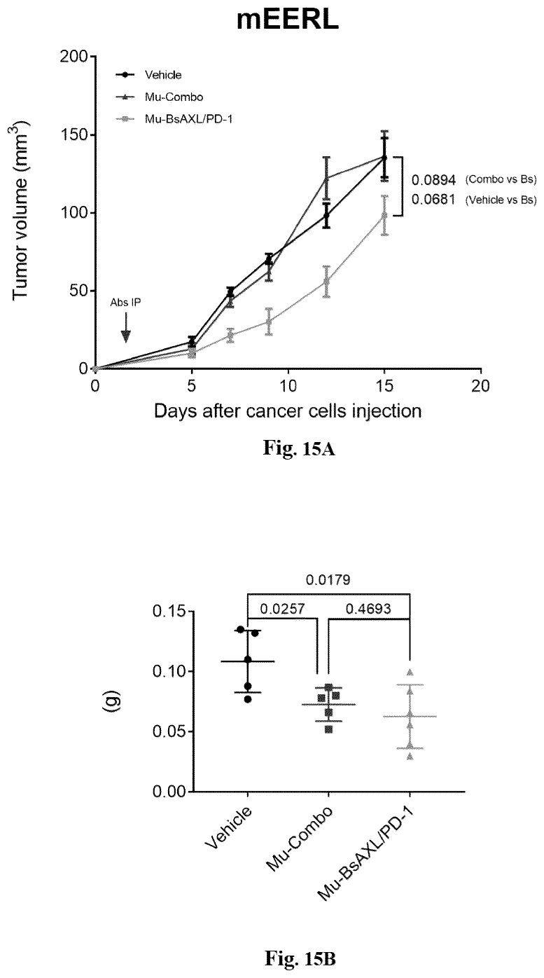

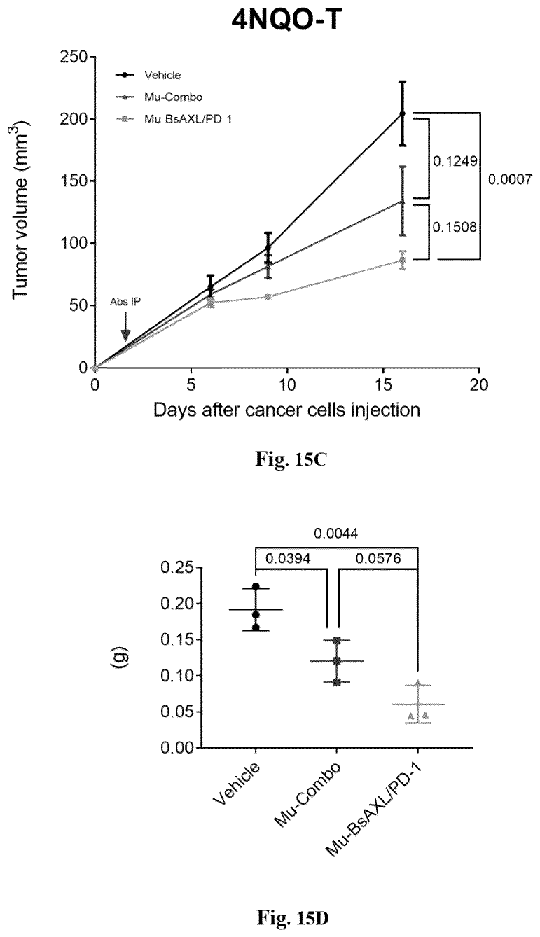

Next, the efficacy of Mu-BsAb-AXL/PD-1 in inhibiting tumor cell growth was tested in comparison with a combination of the two respective mono-specific Abs (Combo). WT mice were infected according to an orthotopic model either with mEERL cancer cells (an HPV-positive HNSCC line) or with 4NQO-T cell line. The mice populations were then treated twice weekly for about two weeks with 5 mg/kg of BsAb, Combo, or a negative control including only Ab vehicle. The results representing the progression of tumor growth are presented in FIGS. 15A-15D. As can be seen in the Figures, in both cancer models the Mu-BsAXL/PD-1 treatment delayed tumor growth, and achieved endpoint tumor weight measurements which were smaller than the endpoint tumor weight achieved by the Combo, and significantly smaller than the tumors of the vehicle treated mice. This indicates a global inhibitory effect of the Bi-3 antibodies on the progression of different types of HNSCCs in vivo, suggesting a potential therapeutic effect of Bi-3.

1. A bi-specific construct comprising two different single-chain variable fragments (scFvs), one targeted to the human receptor AXL and one targeted to human programmed cell death protein 1 (PD1), wherein each of the scFvs comprises a heavy chain variable region (VH) and a light chain variable region (VL) connected, and wherein the two different scFvs are connected to a carrier polypeptide.

2. The bi-specific construct according to claim 1, comprising two scFv molecules targeted to the human receptor AXL and two scFv molecules targeted to human PD1.

3. The bi-specific construct according to claim 1, wherein the two different scFvs and the carrier polypeptide form a structure selected from scFv(PD-1)-carrier-scFv(AXL) and scFv(AXL)-carrier-scFv(PD-1).

4. The bi-specific construct according to claim 1, comprising two polypeptides, each polypeptide comprising two different scFvs and a carrier polypeptide that form a structure selected from scFv(PD-1)-carrier-scFv(AXL) and scFv(AXL)-carrier-scFv(PD-1).

5. The bi-specific construct of claim 1, wherein the carrier polypeptide is connected to the terminal of the scFv-scFv molecules to form a construct selected from the group consisting of: carrier-scFv(AXL)-scFv(PD-1); and scFv(PD-1)-scFv(AXL)-carrier.

6. The bi-specific construct according to claim 1, wherein: (a) the VH and VL of each scFv are connected through a linker of 1-50 amino acid residues; (b) the two different scFvs are connected through a linker of 1-50 amino acid residues to a carrier polypeptide; or, both (a) and (b).

7. The bi-specific construct according to claim 6, wherein at least one of the linkers comprises 2-8 consecutive repeats of the sequence GGGGS (SEQ ID NO: 21).

8. The bi-specific construct according to claim 1, wherein the AXL-targeted scFv comprises the set of six CDR sequences: GSWIH (CDR-H1, SEQ ID NO: 1), WINPYRGYAYYADSVKG (CDR-H2, SEQ ID NO: 2), EYSGWGGSSVGYAMDY (CDR-H3, SEQ ID NO: 3), RASQDVSTA (CDR-L1, SEQ ID NO: 4), SASFLYS (CDR-L2, SEQ ID NO: 5), and QQSYTTPPT (CDR-L3, SEQ ID NO: 6); or comprises the sequence: DIQMTQSPSSLSASVGDRVTITCRASQDVSTAVAWYQQKPGKAPKLLIYSASFLYS GVPSRFSGSGSGTDFTLTISSLQPEDFATYYCQQSYTTPPTFGQGTKVEIKGGGGSG GGGSGGGGSGGGGSEVQLVESGGGLVQPGGSLRLSCAASGFSLSGSWIHWVRQAP GKGLEWVGWINPYRGYAYYADSVKGRFTISADTSKNTAYLQMNSLRAEDTAVYY CAREYSGWGGSSVGYAMDYWGQGTLVGGGGSGGGGSGGGGSGGGGS (SEQ ID NO: 7), or an analog or derivative having at least 90% identity with said sequence.

9. The bi-specific construct according to claim 1, wherein the PD1-targeted scFv comprises the set of six CDRs sequences: NSGMH (CDR-H1, SEQ ID NO: 8), VIWYDGSKRYYADSVKG (CDR-H2, SEQ ID NO: 9), NDDY (CDR-H3, SEQ ID NO: 10), RASQSVSSYLA (CDR-L1, SEQ ID NO: 11), DASNRAT (CDR-L2, SEQ ID NO: 12), and QQSSNWPRT (CDR-L3, SEQ ID NO: 13); or comprises the sequence: EIVLTQSPATLSLSPGERATLSCRASQSVSSYLAWYQQKPGQAPRLLIYDASNRATG IPARFSGSGSGTDFTLTISSLEPEDFAVYYCQQSSNWPRTFGQGTKVEIKGGGGSGG GGSGGGGSGGGGSQVQLVESGGGVVQPGRSLRLDCKASGITFSNSGMHWVRQAP GKGLEWVAVIWYDGSKRYYADSVKGRFTISRDNSKNTLFLQMNSLRAEDTAVYY CATNDDYWGQGTLVTVSSGGGGSGGGGSGGGGSGGGGS (SEQ ID NO: 14), or an analog or derivative having at least 90% identity with said sequence.

10. The bi-specific construct according to claim 1, wherein the PD1-targeted scFv, comprises the sequence: EIVLTQSPATLSLSPGERATLSCRASQSVSSYLAWYQQKPGQAPRLLIYDASNRATG IPARFSGSGSGTDFTLTISSLEPEDFAVYYCQQSSNWPRTFGQGTKVEIKGGGGSGG GGSGGGGSGGGGSQVQLVESGGGVVQPGRSLRLDCKASGITFSNSGMHWVRQAP GKGLEWVAVIWYDGSKRYYADSVKGRFTISRDNSKNTLFLQMNSLRAEDTAVYY CATNDDYWGQGTLVTVSSGGGGSGGGGSGGGGSGGGGS (SEQ ID NO: 14); and the AXL-targeted scFv, comprises the sequence: DIQMTQSPSSLSASVGDRVTITCRASQDVSTAVAWYQQKPGKAPKLLIYSASFLYS GVPSRFSGSGSGTDFTLTISSLQPEDFATYYCQQSYTTPPTFGQGTKVEIKGGGGSG GGGSGGGGSGGGGSEVQLVESGGGLVQPGGSLRLSCAASGFSLSGSWIHWVRQAP GKGLEWVGWINPYRGYAYYADSVKGRFTISADTSKNTAYLQMNSLRAEDTAVYY CAREYSGWGGSSVGYAMDYWGQGTLVGGGGSGGGGSGGGGSGGGGS (SEQ ID NO: 7), or an analog or derivative having at least 90% identity with any of said sequences.

11. The bi-specific construct according to claim 1, wherein the carrier polypeptide sequence comprises a human IgG1 constant domain (Fc) or a fragment thereof.

12. The bi-specific construct according to claim 11, comprising two polypeptides, each polypeptide comprising two different scFvs and a carrier polypeptide, wherein the bi-specific construct forms a structure selected from [scFv(PD-1)]₂-IgG1Fc-[scFv(AXL)]₂ and [scFv(AXL)]₂-IgG1Fc-[scFv(PD-1)]₂.

13. The bi-specific construct according to claim 1, wherein the construct comprises a sequence selected from the group consisting of: PD1-Fc-AXL (SEQ ID NO: 24) and PD1-AXL-Fc (SEQ ID NO: 22).

14. A nucleic acid sequence encoding a bi-specific construct according to claim 1 or encoding at least one portion of said bi-specific construct.

15. The nucleic acid sequence according to claim 14, selected from the group consisting of:
(i) a nucleic acid sequence that encodes AXL-targeted scFv (VL-(GGGGS)₄—VH-(GGGGS)₄), wherein the nucleic acid sequence comprises SEQ ID NO: 16;
(ii) a nucleic acid sequence that encodes a PD1-targeted scFv (VL-(GGGGS)₄—VH-(GGGGS)₄), wherein the nucleic acid sequence comprises SEQ ID NO: 17;
(iii) a nucleic acid sequence that encodes the PD1-Fc-AXL bi-specific construct of SEQ ID NO: 24;
(iv) a nucleic acid sequence that encodes the PD1-AXL-Fc bi-specific construct of SEQ ID NO: 22;
(v) a nucleic acid sequence that encodes the bi-specific construct denoted bi-3 (PD1-Fc-AXL), wherein the nucleic acid sequence comprises SEQ ID NO: 20;
(vi) a nucleic acid sequence that encodes the bi-specific construct denoted bi-1 (PD1-AXL-Fc), wherein the nucleic acid sequence comprises SEQ ID NO: 18; and
(vii) a nucleic acid sequence having at least 80% identity with any of said sequences.

16. A vector comprising a nucleic acid according to claim 14.

17. A pharmaceutical composition comprising at least one bi-specific construct according to claim 1, and a pharmaceutically acceptable excipient, diluent, salt or buffer.

18. A method for inhibiting the growth or proliferation of cancer cells or for promoting T cell-mediated killing of the cancer cells, comprising contacting the cancer cells with the bi-specific constructs according to claim 1.

19. A method for preventing, attenuating or treating cancer, comprising administering to a subject in need thereof, the bi-specific construct according to claim 1.

20. The method according to claim 19, wherein the cancer is a cancer that expresses AXL and/or PD1, optionally head and neck carcinoma (HNSCC) or esophageal squamous cell carcinoma (ESCC).


SEQUENCE LISTING (27 sequences)

SEQ ID NO: 1 - protein, 5 aa, synthetic construct
GSWIH

SEQ ID NO: 2 - protein, 17 aa, synthetic construct
WINPYRGYAYYADSVKG

SEQ ID NO: 3 - protein, 16 aa, synthetic construct
EYSGWGGSSVGYAMDY

SEQ ID NO: 4 - protein, 9 aa, synthetic construct
RASQDVSTA

SEQ ID NO: 5 - protein, 7 aa, synthetic construct
SASFLYS

SEQ ID NO: 6 - protein, 9 aa, synthetic construct
QQSYTTPPT

SEQ ID NO: 7 - protein, 268 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVGGGGSGGGGSGGGGSGGGGS

SEQ ID NO: 8 - protein, 5 aa, synthetic construct
NSGMH

SEQ ID NO: 9 - protein, 17 aa, synthetic construct
VIWYDGSKRYYADSVKG

SEQ ID NO: 10 - protein, 4 aa, synthetic construct
NDDY

SEQ ID NO: 11 - protein, 11 aa, synthetic construct
RASQSVSSYLA

SEQ ID NO: 12 - protein, 7 aa, synthetic construct
DASNRAT

SEQ ID NO: 13 - protein, 9 aa, synthetic construct
QQSSNWPRT

SEQ ID NO: 14 - protein, 260 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGS

SEQ ID NO: 15 - DNA, 669 bp, synthetic construct
  source 1..669: mol_type = other DNA
gtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaa

SEQ ID NO: 16 - DNA, 5004 bp, synthetic construct
  source 1..5004: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatatcggccatggatgacatccaaatgactcaaagcccatcctctctctctgcgagcgtgggcgacagggtgacaatcacatgccgggcaagccaagatgtttccactgcagtggcatggtaccaacaaaagcccggtaaagcaccaaaactcctcatatactctgcaagctttctctactctggtgtgccttctcgcttcagcggttccggcagcggtacagatttcaccctcacaatttcttctctccaacctgaagatttcgccacatactactgccaacaatcttatacaacaccccctacattcggccaaggcactaaagttgagataaaaggaggaggaggcagcggaggcggcggttctggaggtggaggtagtggtggcggtggttctgaagtccaactggttgaaagcggcggcggactggtccaacccggcggcagtctccgcctgagttgcgcagcctccggtttttccctgtccggcagctggattcactgggttagacaagctcctggaaaaggcctcgagtgggttggttggataaacccatatcgcggatacgcatattacgcagactctgtcaaaggtcggtttaccatcagcgccgacacaagcaagaatactgcatacctgcaaatgaacagcctgagggctgaggataccgctgtttattactgtgcaagagaatactctggatggggtggctctagcgtcggttacgcaatggattactggggccaaggtactttagttggtggtggaggttcgggaggaggtggatcaggaggaggaggatccggtggtggaggtagtagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaacatcaccatcaccatcactgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 17 - DNA, 4980 bp, synthetic construct
  source 1..4980: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatatcggccatggatgaaattgtcctcacccaaagccctgcaactctgtctctcagccctggcgaacgcgcaaccctgtcttgtagagcatctcaaagcgtgtccagctatctcgcctggtaccagcagaagcccgggcaagctccacggctcctgatatacgacgcatccaaccgcgcaactgggattcctgcacggttttccggaagcggctccgggaccgattttacactcacaatatcctccctcgagccagaggacttcgctgtttactactgccagcaatccagcaactggcctagaacctttggacagggcacaaaagttgagataaaagggggcgggggctccggcggaggcggttccggcgggggtgggagcggcggaggtggatctcaggtgcaactggttgaatctggaggaggcgtcgttcaaccaggacggagcctcagactggactgcaaggcctctgggattactttttccaactccgggatgcattgggtgcgccaggccccaggcaagggcctggagtgggttgccgtgatctggtacgatggtagcaaacgctattacgccgactccgttaaaggcagatttaccatttctcgcgataattccaaaaacaccctcttcctccagatgaattccctccgcgccgaagacacagccgtttactactgcgctactaatgatgattactggggtcagggtactctggtgacagtgtcttctggtggtggaggttcgggaggaggtggatcaggaggaggaggatccggtggtggaggtagtagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaacatcaccatcaccatcactgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 18 - DNA, 5784 bp, synthetic construct
  source 1..5784: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatatcggccatggatgaaattgtcctcacccaaagccctgcaactctgtctctcagccctggcgaacgcgcaaccctgtcttgtagagcatctcaaagcgtgtccagctatctcgcctggtaccagcagaagcccgggcaagctccacggctcctgatatacgacgcatccaaccgcgcaactgggattcctgcacggttttccggaagcggctccgggaccgattttacactcacaatatcctccctcgagccagaggacttcgctgtttactactgccagcaatccagcaactggcctagaacctttggacagggcacaaaagttgagataaaagggggcgggggctccggcggaggcggttccggcgggggtgggagcggcggaggtggatctcaggtgcaactggttgaatctggaggaggcgtcgttcaaccaggacggagcctcagactggactgcaaggcctctgggattactttttccaactccgggatgcattgggtgcgccaggccccaggcaagggcctggagtgggttgccgtgatctggtacgatggtagcaaacgctattacgccgactccgttaaaggcagatttaccatttctcgcgataattccaaaaacaccctcttcctccagatgaattccctccgcgccgaagacacagccgtttactactgcgctactaatgatgattactggggtcagggtactctggtgacagtgtcttctggtggtggaggttcgggaggaggtggatcaggaggaggaggatccggtggtggaggtagtgacatccaaatgactcaaagcccatcctctctctctgcgagcgtgggcgacagggtgacaatcacatgccgggcaagccaagatgtttccactgcagtggcatggtaccaacaaaagcccggtaaagcaccaaaactcctcatatactctgcaagctttctctactctggtgtgccttctcgcttcagcggttccggcagcggtacagatttcaccctcacaatttcttctctccaacctgaagatttcgccacatactactgccaacaatcttatacaacaccccctacattcggccaaggcactaaagttgagataaaaggaggaggaggcagcggaggcggcggttctggaggtggaggtagtggtggcggtggttctgaagtccaactggttgaaagcggcggcggactggtccaacccggcggcagtctccgcctgagttgcgcagcctccggtttttccctgtccggcagctggattcactgggttagacaagctcctggaaaaggcctcgagtgggttggttggataaacccatatcgcggatacgcatattacgcagactctgtcaaaggtcggtttaccatcagcgccgacacaagcaagaatactgcatacctgcaaatgaacagcctgagggctgaggataccgctgtttattactgtgcaagagaatactctggatggggtggctctagcgtcggttacgcaatggattactggggccaaggtactttagttggtggtggaggttcgggaggaggtggatcaggaggaggaggatccggtggtggaggtagtagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaacatcaccatcaccatcactgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 19 - DNA, 5784 bp, synthetic construct
  source 1..5784: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatatcggccatggatgacatccaaatgactcaaagcccatcctctctctctgcgagcgtgggcgacagggtgacaatcacatgccgggcaagccaagatgtttccactgcagtggcatggtaccaacaaaagcccggtaaagcaccaaaactcctcatatactctgcaagctttctctactctggtgtgccttctcgcttcagcggttccggcagcggtacagatttcaccctcacaatttcttctctccaacctgaagatttcgccacatactactgccaacaatcttatacaacaccccctacattcggccaaggcactaaagttgagataaaaggaggaggaggcagcggaggcggcggttctggaggtggaggtagtggtggcggtggttctgaagtccaactggttgaaagcggcggcggactggtccaacccggcggcagtctccgcctgagttgcgcagcctccggtttttccctgtccggcagctggattcactgggttagacaagctcctggaaaaggcctcgagtgggttggttggataaacccatatcgcggatacgcatattacgcagactctgtcaaaggtcggtttaccatcagcgccgacacaagcaagaatactgcatacctgcaaatgaacagcctgagggctgaggataccgctgtttattactgtgcaagagaatactctggatggggtggctctagcgtcggttacgcaatggattactggggccaaggtactttagttggtggtggaggttcgggaggaggtggatcaggaggaggaggatccggtggtggaggtagtgaaattgtcctcacccaaagccctgcaactctgtctctcagccctggcgaacgcgcaaccctgtcttgtagagcatctcaaagcgtgtccagctatctcgcctggtaccagcagaagcccgggcaagctccacggctcctgatatacgacgcatccaaccgcgcaactgggattcctgcacggttttccggaagcggctccgggaccgattttacactcacaatatcctccctcgagccagaggacttcgctgtttactactgccagcaatccagcaactggcctagaacctttggacagggcacaaaagttgagataaaagggggcgggggctccggcggaggcggttccggcgggggtgggagcggcggaggtggatctcaggtgcaactggttgaatctggaggaggcgtcgttcaaccaggacggagcctcagactggactgcaaggcctctgggattactttttccaactccgggatgcattgggtgcgccaggccccaggcaagggcctggagtgggttgccgtgatctggtacgatggtagcaaacgctattacgccgactccgttaaaggcagatttaccatttctcgcgataattccaaaaacaccctcttcctccagatgaattccctccgcgccgaagacacagccgtttactactgcgctactaatgatgattactggggtcagggtactctggtgacagtgtcttctggtggtggaggttcgggaggaggtggatcaggaggaggaggatccggtggtggaggtagtagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaacatcaccatcaccatcactgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 20 - DNA, 5784 bp, synthetic construct
  source 1..5784: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatatcggccatggatgaaattgtcctcacccaaagccctgcaactctgtctctcagccctggcgaacgcgcaaccctgtcttgtagagcatctcaaagcgtgtccagctatctcgcctggtaccagcagaagcccgggcaagctccacggctcctgatatacgacgcatccaaccgcgcaactgggattcctgcacggttttccggaagcggctccgggaccgattttacactcacaatatcctccctcgagccagaggacttcgctgtttactactgccagcaatccagcaactggcctagaacctttggacagggcacaaaagttgagataaaagggggcgggggctccggcggaggcggttccggcgggggtgggagcggcggaggtggatctcaggtgcaactggttgaatctggaggaggcgtcgttcaaccaggacggagcctcagactggactgcaaggcctctgggattactttttccaactccgggatgcattgggtgcgccaggccccaggcaagggcctggagtgggttgccgtgatctggtacgatggtagcaaacgctattacgccgactccgttaaaggcagatttaccatttctcgcgataattccaaaaacaccctcttcctccagatgaattccctccgcgccgaagacacagccgtttactactgcgctactaatgatgattactggggtcagggtactctggtgacagtgtcttctggtggtggaggttcgggaggaggtggatcaggaggaggaggatccggtggtggaggtagtagatctgtggagtgcccaccttgcccagcaccacctgtggcaggaccttcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaaggtggtggaggttcgggaggaggtggatcaggaggaggaggatccggtggtggaggtagtgacatccaaatgactcaaagcccatcctctctctctgcgagcgtgggcgacagggtgacaatcacatgccgggcaagccaagatgtttccactgcagtggcatggtaccaacaaaagcccggtaaagcaccaaaactcctcatatactctgcaagctttctctactctggtgtgccttctcgcttcagcggttccggcagcggtacagatttcaccctcacaatttcttctctccaacctgaagatttcgccacatactactgccaacaatcttatacaacaccccctacattcggccaaggcactaaagttgagataaaaggaggaggaggcagcggaggcggcggttctggaggtggaggtagtggtggcggtggttctgaagtccaactggttgaaagcggcggcggactggtccaacccggcggcagtctccgcctgagttgcgcagcctccggtttttccctgtccggcagctggattcactgggttagacaagctcctggaaaaggcctcgagtgggttggttggataaacccatatcgcggatacgcatattacgcagactctgtcaaaggtcggtttaccatcagcgccgacacaagcaagaatactgcatacctgcaaatgaacagcctgagggctgaggataccgctgtttattactgtgcaagagaatactctggatggggtggctctagcgtcggttacgcaatggattactggggccaaggtactttagttcatcaccatcaccatcactgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 21 - protein, 5 aa, synthetic construct
GGGGS

SEQ ID NO: 22 - protein, 753 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVGGGGSGGGGSGGGGSGGGGSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 23 - protein, 753 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 24 - protein, 753 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLV

SEQ ID NO: 25 - protein, 759 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVGGGGSGGGGSGGGGSGGGGSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKHHHHHH

SEQ ID NO: 26 - protein, 759 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKHHHHHH

SEQ ID NO: 27 - protein, 759 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKGGGGSGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSGGGGSRSVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSGGGGSEVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVHHHHHH